(19) United States
(12) Patent Application Publication
(10) Pub. No.: US 2020/0005927 A1
(43) Pub. Date: Jan. 2, 2020

(54) Digital Audio/Visual Processing System and Method

(71) Applicant: Solstice Strategy Partners, LLC, Tolland, CT (US)

(72) Inventors: Delanea Anne Davis, Tolland, CT (US); Rita Faith MacRae, South Windsor, CT (US)

(21) Appl. No.: 16/570,847

(22) Filed: Sep. 13, 2019

Related U.S. Application Data
(63) Continuation of application No. 15/395,681, filed on Dec. 30, 2016.
(60) Provisional application No. 62/273,513, filed on Dec. 31, 2015.

(51) Int. Cl. G16H 20/40; G16H 40/63

(52) U.S. Cl. CPC G16H 20/40; G16H 40/63

(57) ABSTRACT

A computer-implemented digital audio/visual processing system and method includes receiving digital audio/visual sensory component files having various file types (script/words, music/tones, beats/syncopation, sound waves, images/video), each of the digital audio/visual sensory component files comprising detailed audio/visual sub-component data associated therewith; receiving user data indicative of a user's medical condition, current medical treatment and personal characteristics, the medical condition comprising pain and the current medical treatment comprising pain medication; providing selection factors/attributes based on the user data for each audio/visual sensory component digital file and corresponding audio/visual sub-component data; automatically selecting certain of the audio/visual sensory component digital files based on the selection factors/attributes; automatically combining selected audio/visual sensory component digital files to create a digital audio/visual meditation file; receiving results/outcomes data indicative of results/outcomes for the user and for other users; continuously adjusting the selecting based on the results/outcomes data; and providing the digital audio/visual meditation file to the user.

### CROSS REFERENCES TO RELATED APPLICATIONS

This application is a continuation of U.S. patent application Ser. No. 15/395,681, filed Dec. 30, 2016, which claims the benefit of U.S. Provisional Application Ser. No. 62/273,513, filed Dec. 31, 2015, the entire disclosure of each application referred to above are incorporated herein by reference to the extent permitted by applicable law.

### BACKGROUND

It is common practice to use Reiki, guided meditation, hypnosis, and/or other energy-based techniques to attempt to relax and/or heal the body. Such techniques are often referred to as “Complementary” and/or “Alternative” medicine, or “CAM” to differentiate it from “traditional” medicine practiced by licensed medical doctors, surgeons, nurses, and the like, in hospitals, medical offices, and other clinical settings. However, such techniques typically require a trained professional or practitioner to provide the service to the patient or client, which may limit access to such services for some people due to accessibility or cost of the service. Also, such techniques are often performed using a standard set of treatment steps for all patients, which can result in less-than-optimal or inconsistent outcomes or results for patients.

It is also becoming more common for hospitals to incorporate such CAM techniques and practices into the practice of traditional medical treatment, as it has been found that combining the traditional and CAM approaches to treatment can lead to improved patient outcomes, such as reduced pain and accelerated healing. When such hybrid approaches are effective, the resulting improved outcomes can greatly reduce hospital stay time, as well as re-admissions, thereby reducing overall health care costs.

However, such combined or hybrid treatment approaches are in their infancy and are often done as a standard/uniform “one-size-fits-all”, or “bolt-on” supplement to existing traditional medical treatment. For example, hospitals may provide a general room or area for meditation or other alternative treatments for patients who wish to participate in CAM techniques; but not a coordinated approach to integrate or optimize the treatment approaches. Accordingly, such approaches can also result in inconsistent results for patients.

Thus, it would be desirable to have a system or method that improves the short-comings of existing techniques and that enables improved CAM techniques to provide better patient benefits and outcomes. It would also be desirable to have a system or method that improves hybrid CAM/traditional medical treatment approaches and outcomes.

### BRIEF DESCRIPTION OF THE DRAWINGS

FIG. 1 is a top level block diagram of components of a system and method for computer-controlled adaptable audio/visual treatment, in accordance with embodiments of the present disclosure.

FIG. 1A is a block diagram of various components of the system of FIG. 1, connected via a network, in accordance with embodiments of the present disclosure

FIG. 2 is a top level block diagram showing certain components and flow of data, in accordance with embodiments of the present disclosure.

FIG. 3 is a top level block diagram showing certain components and flow of data, in accordance with embodiments of the present disclosure.

FIG. 4 is a top level block diagram showing certain components and flow of data, in accordance with embodiments of the present disclosure.

FIG. 5 is a top level block diagram showing certain components and flow of data, in accordance with embodiments of the present disclosure.

FIG. 6 is a top level block diagram showing certain components and flow of data, in accordance with embodiments of the present disclosure.

FIG. 7 is a top level block diagram showing certain components and flow of data, in accordance with embodiments of the present disclosure.

FIG. 8 is a top level block diagram showing certain components and flow of data, in accordance with embodiments of the present disclosure.

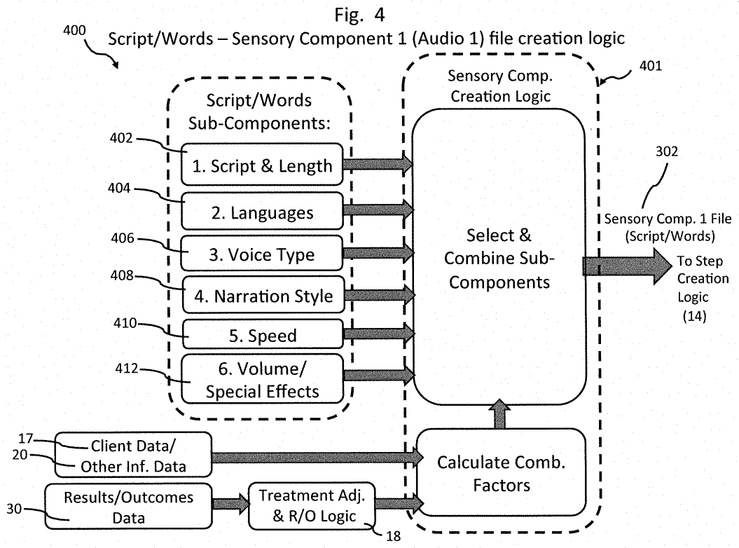

FIG. 4A is an illustration of a digital word/file data structure for audio and video files, in accordance with embodiments of the present disclosure.

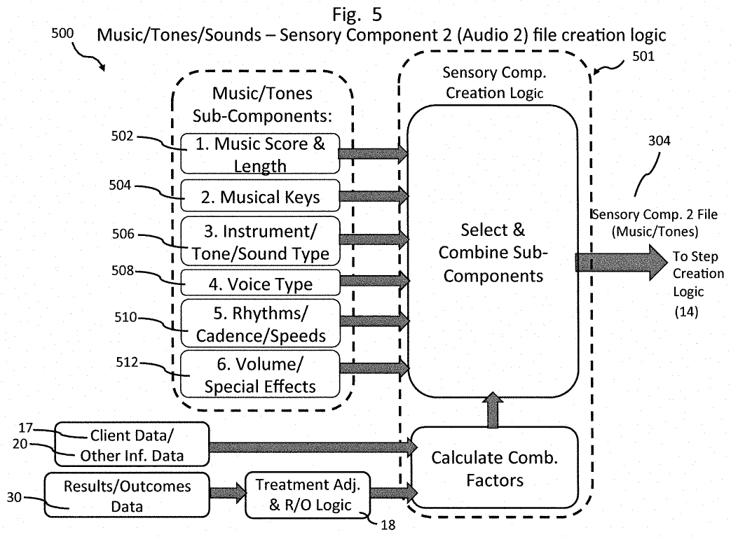

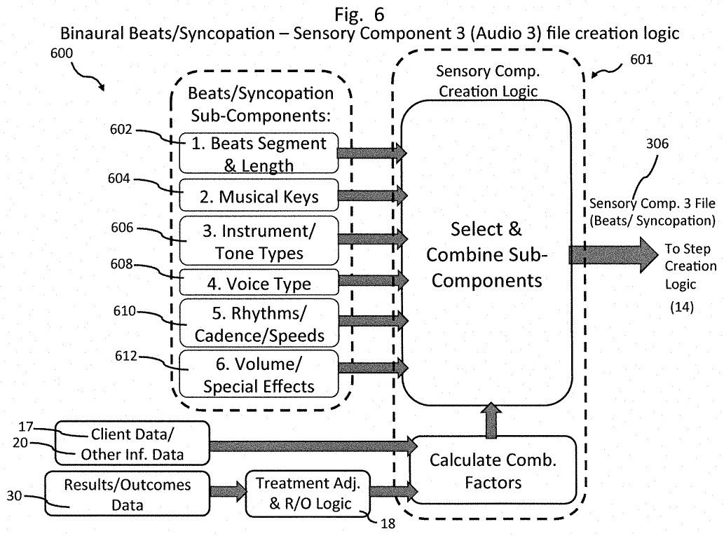

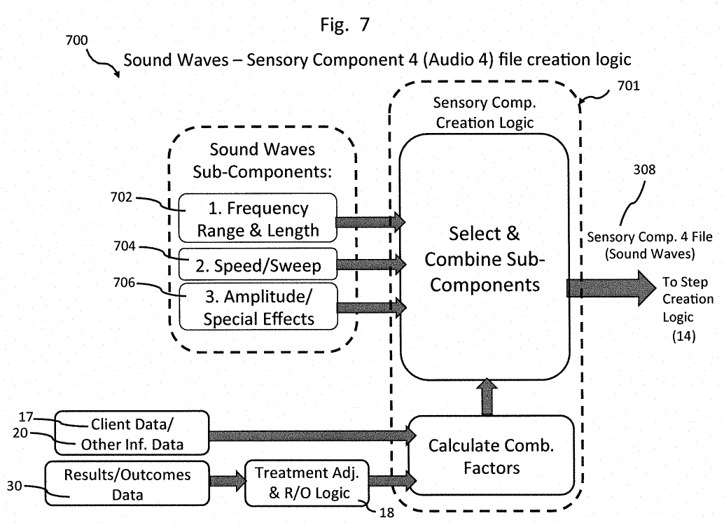

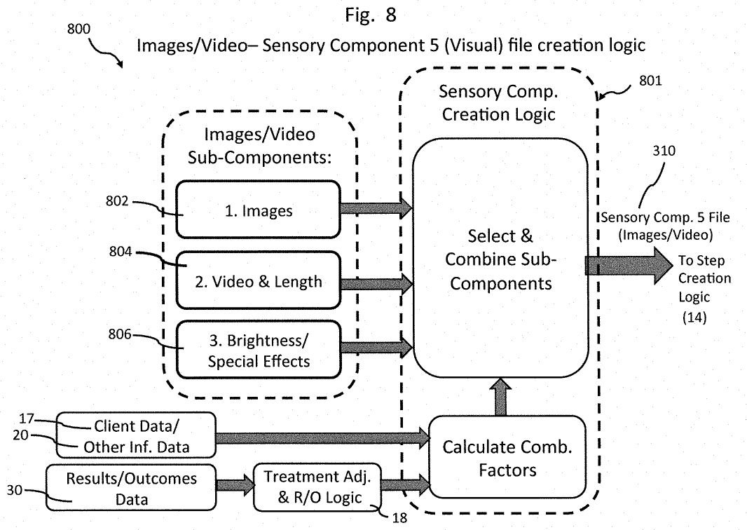

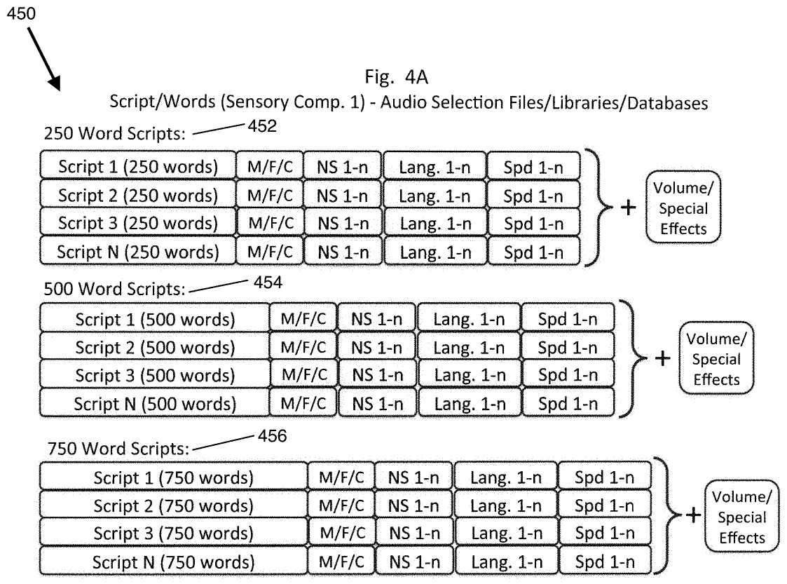

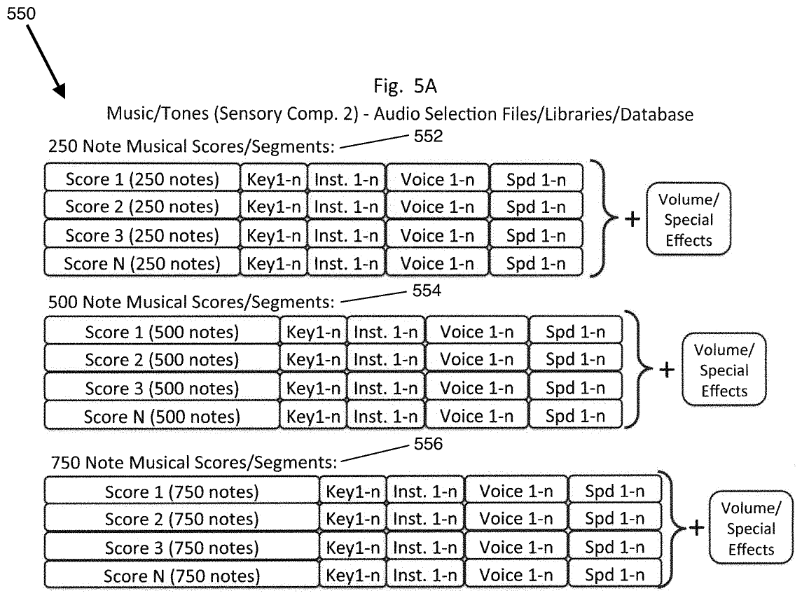

FIG. 5A is an illustration of a digital word/file data structure for audio and video files, in accordance with embodiments of the present disclosure.

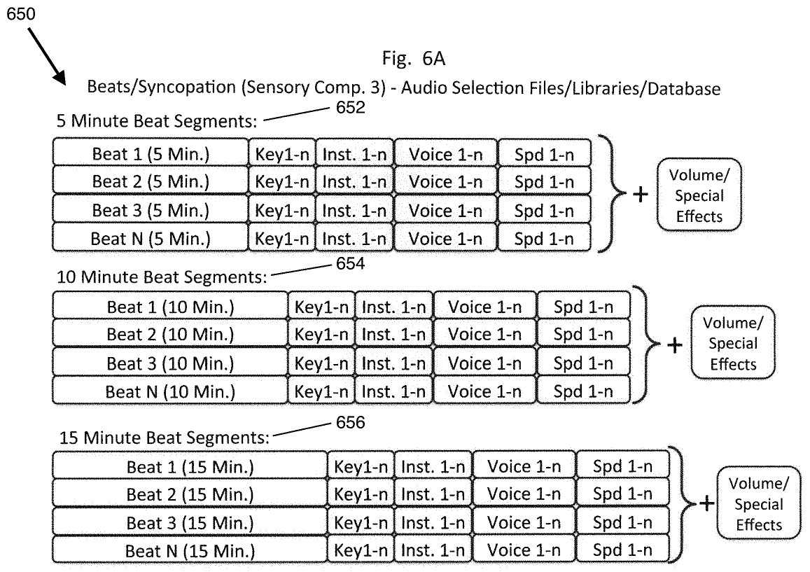

FIG. 6A is an illustration of a digital word/file data structure for audio and video files, in accordance with embodiments of the present disclosure.

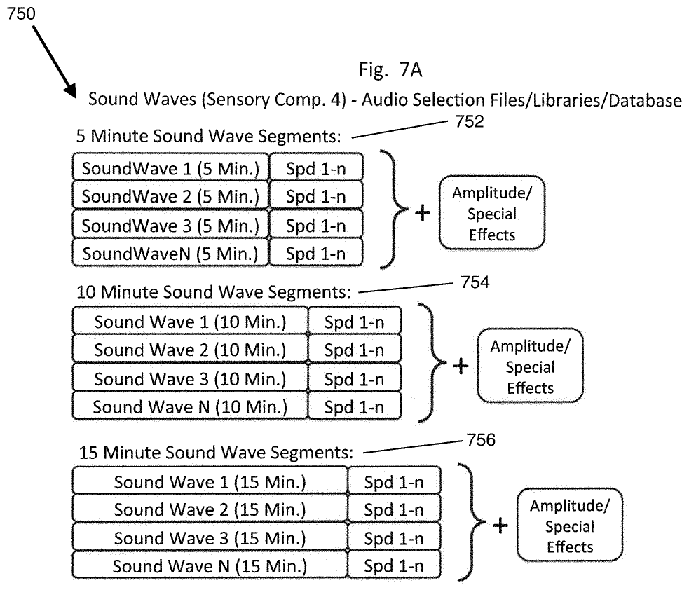

FIG. 7A is an illustration of a digital word/file data structure for audio and video files, in accordance with embodiments of the present disclosure.

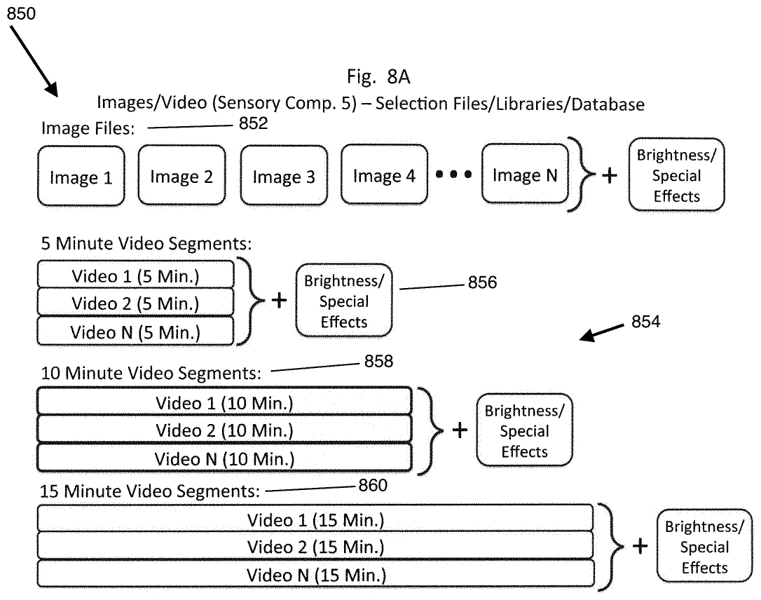

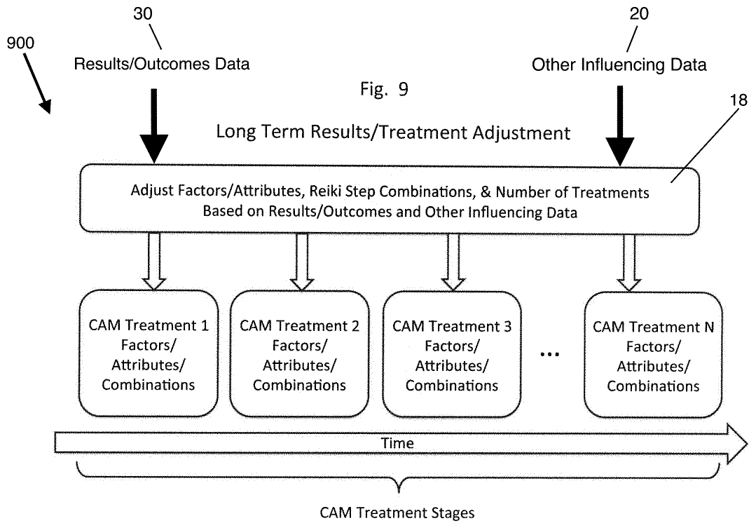

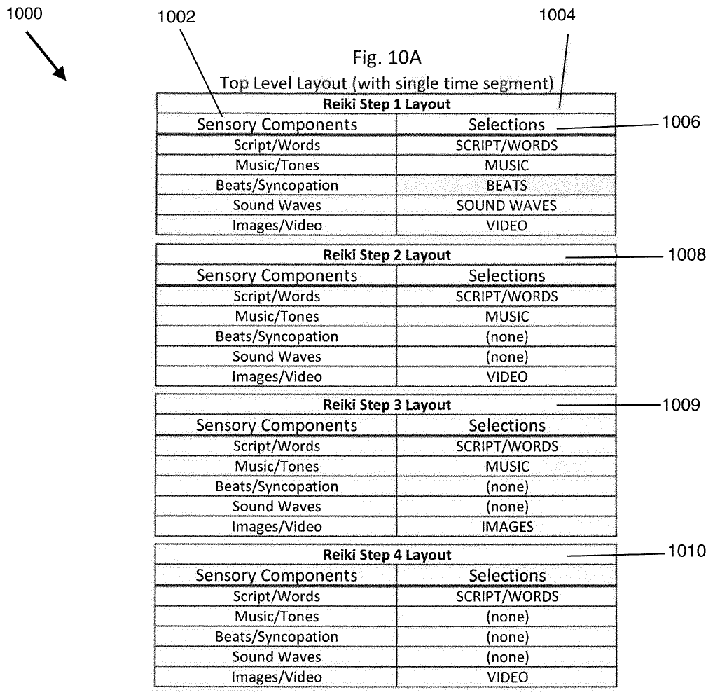

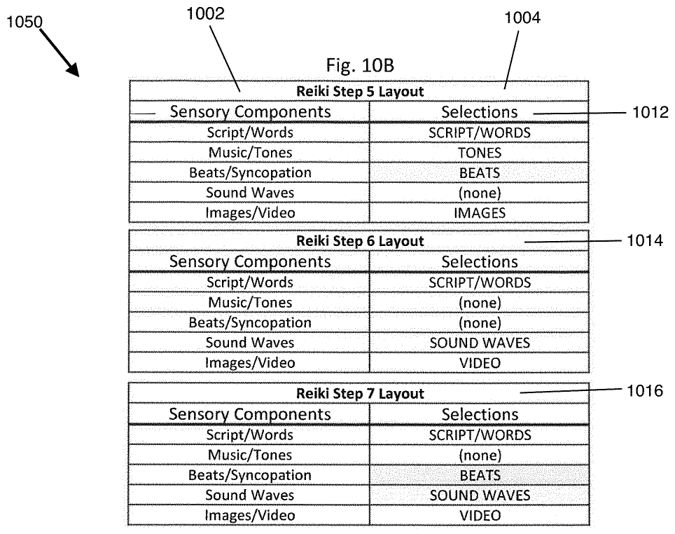

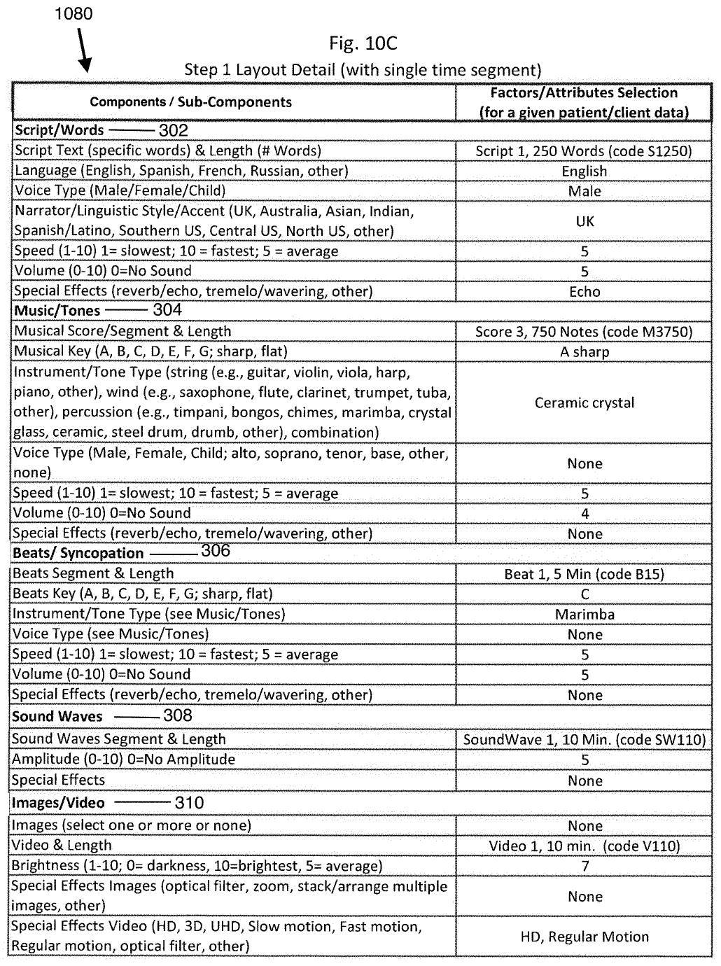

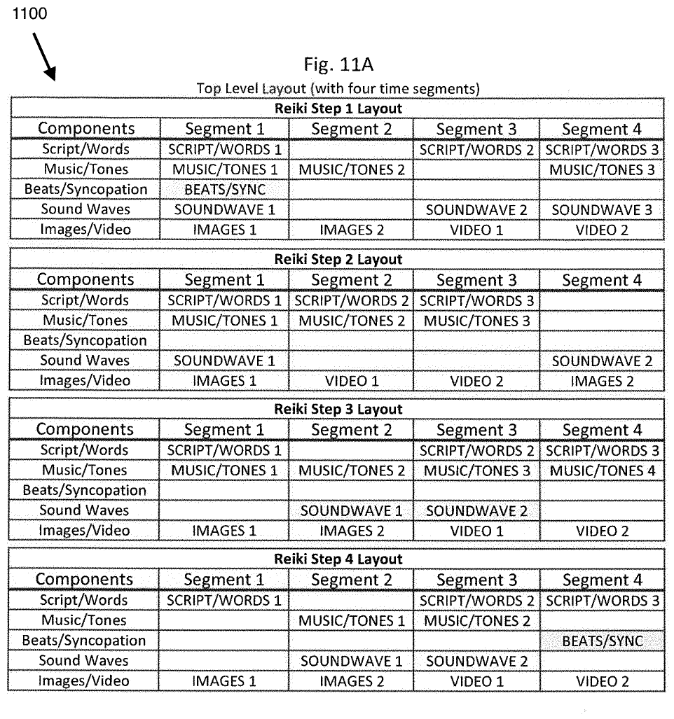

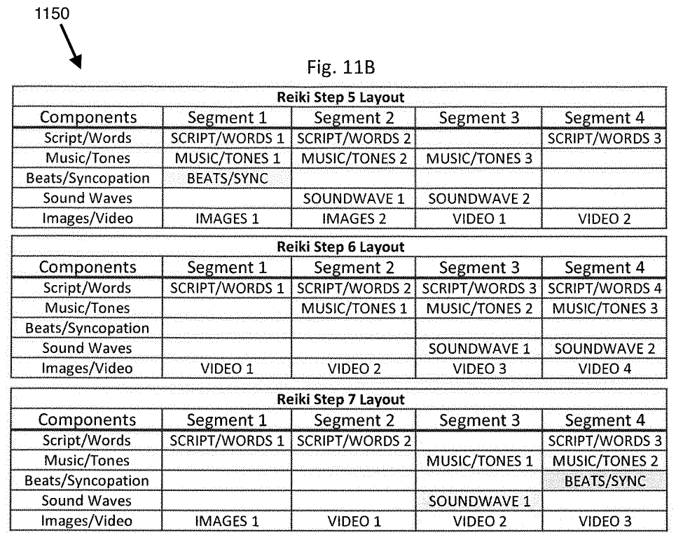

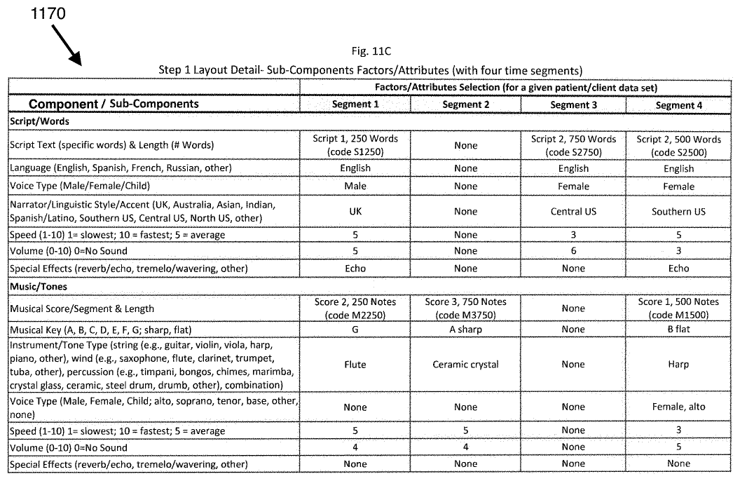

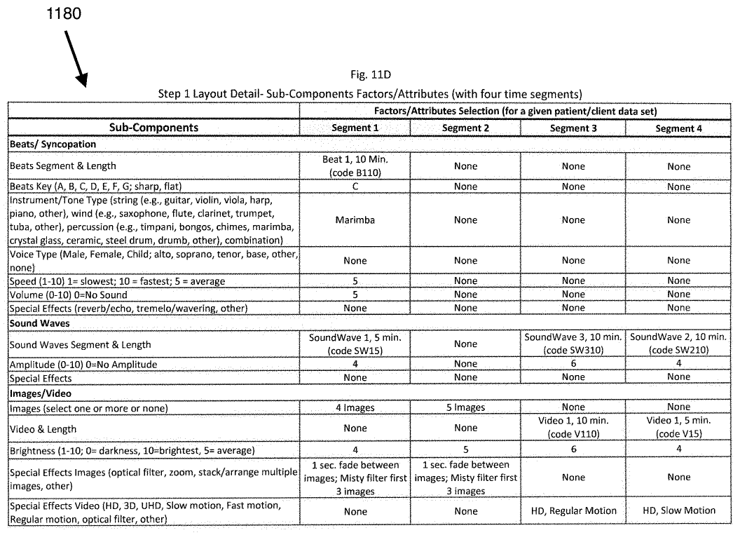

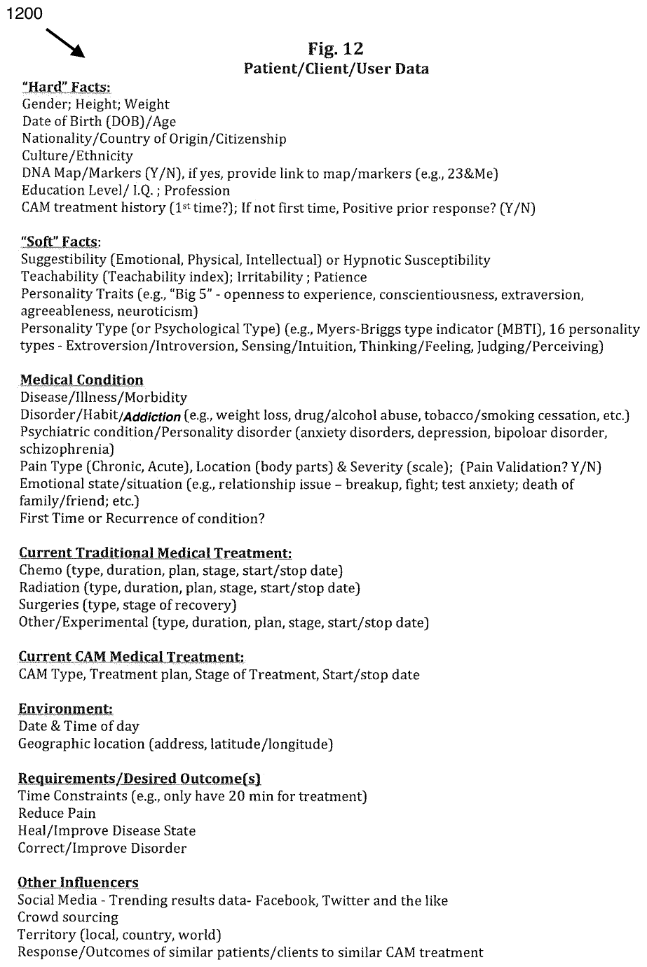

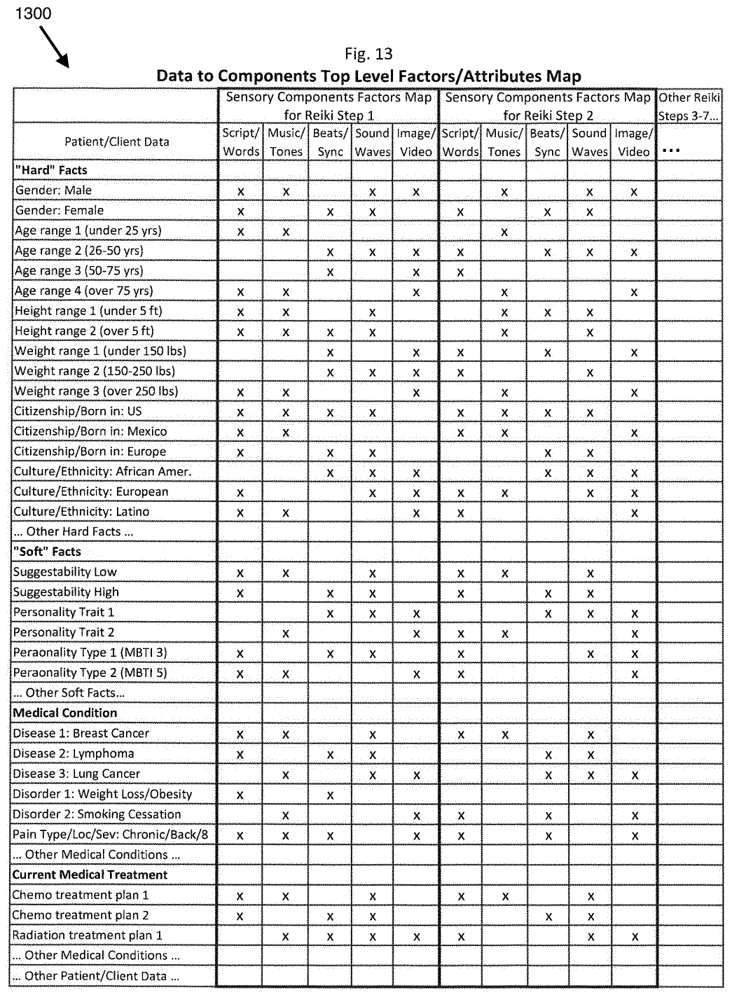

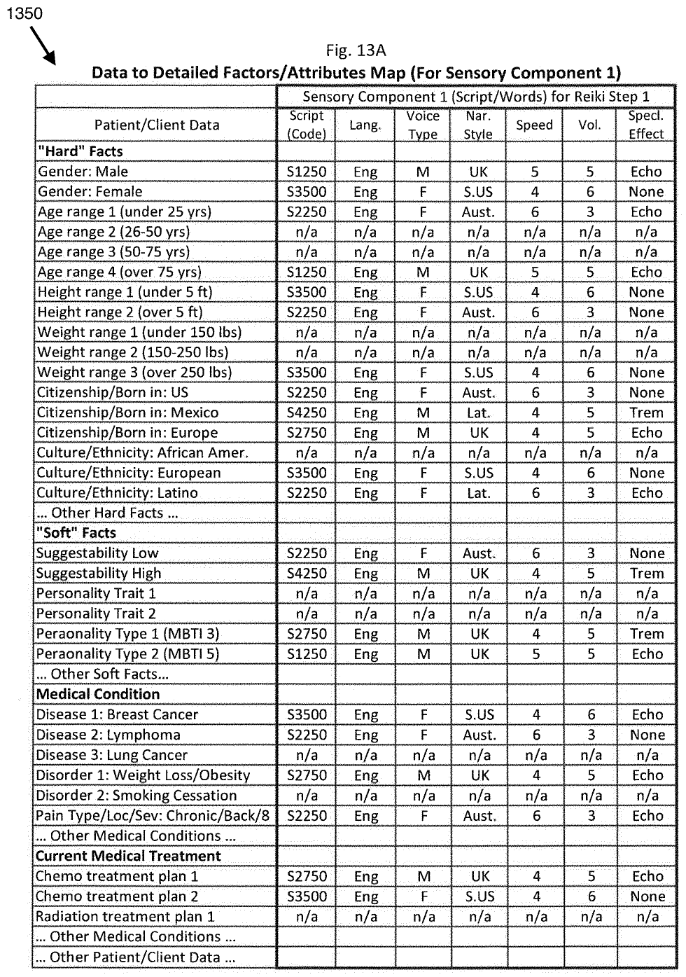

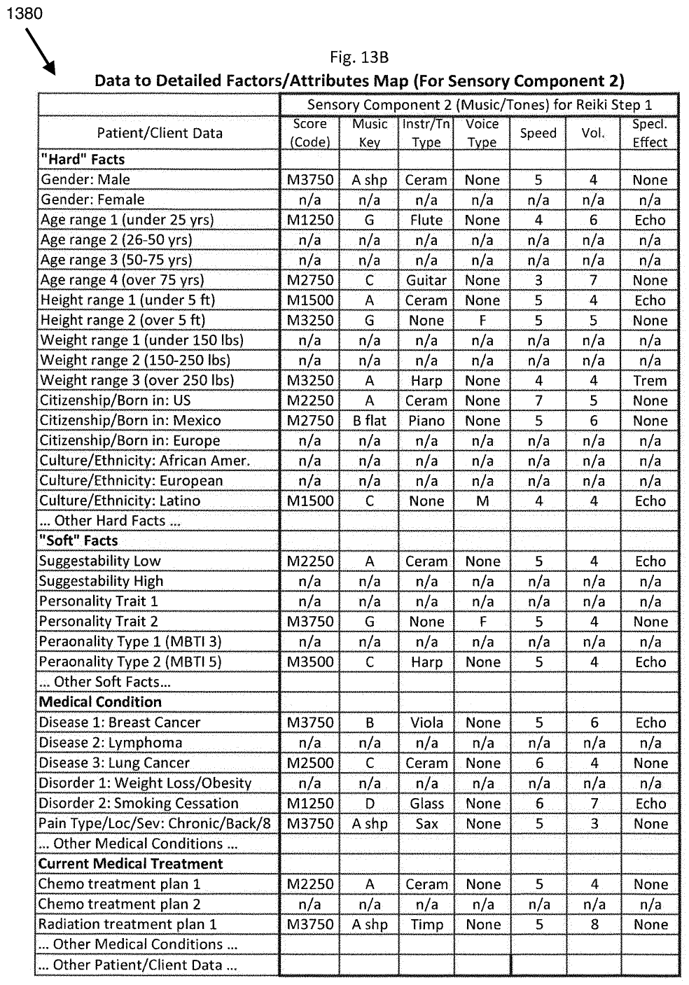

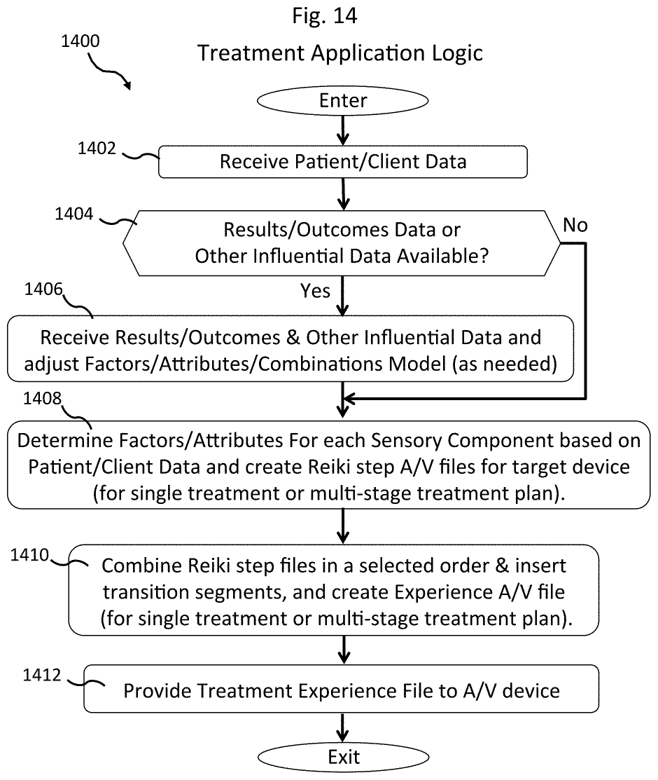

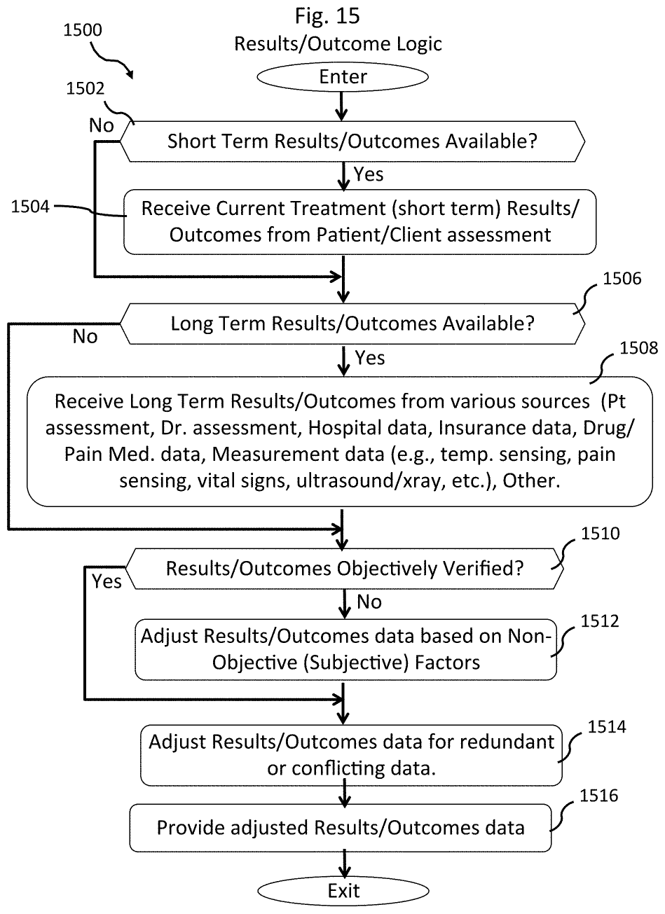

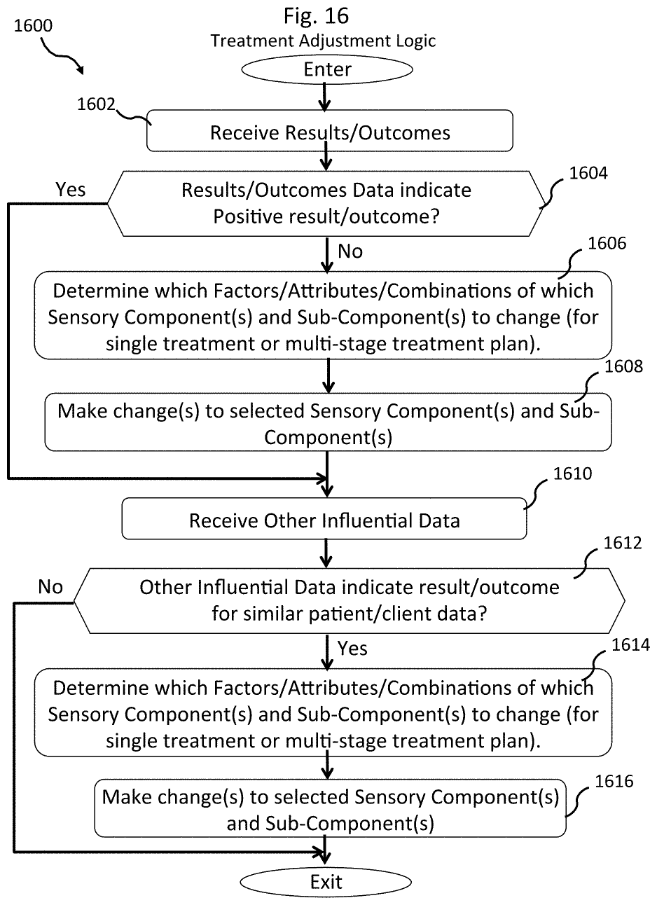

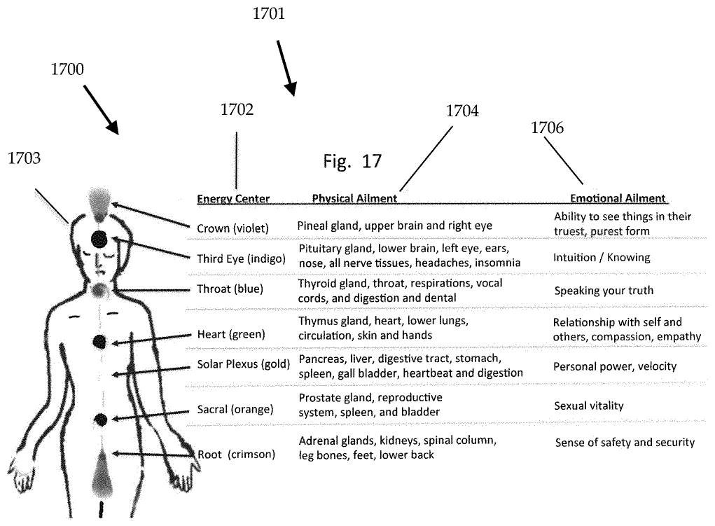

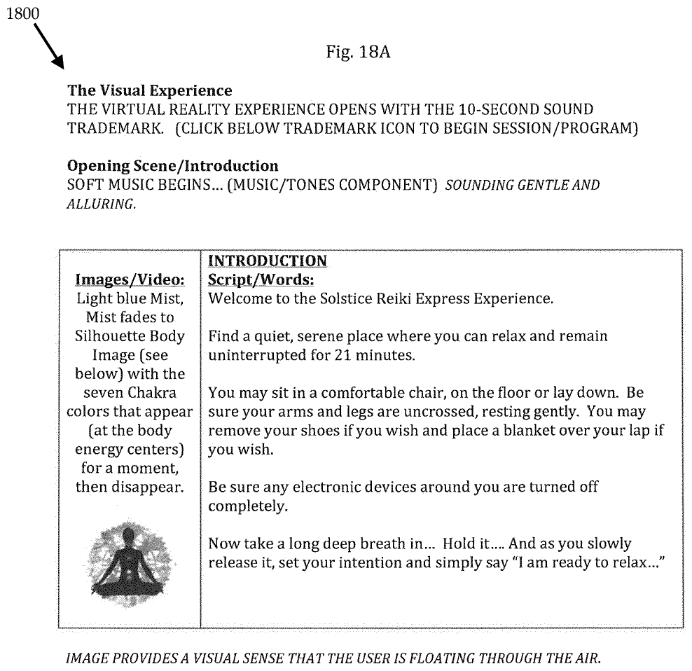

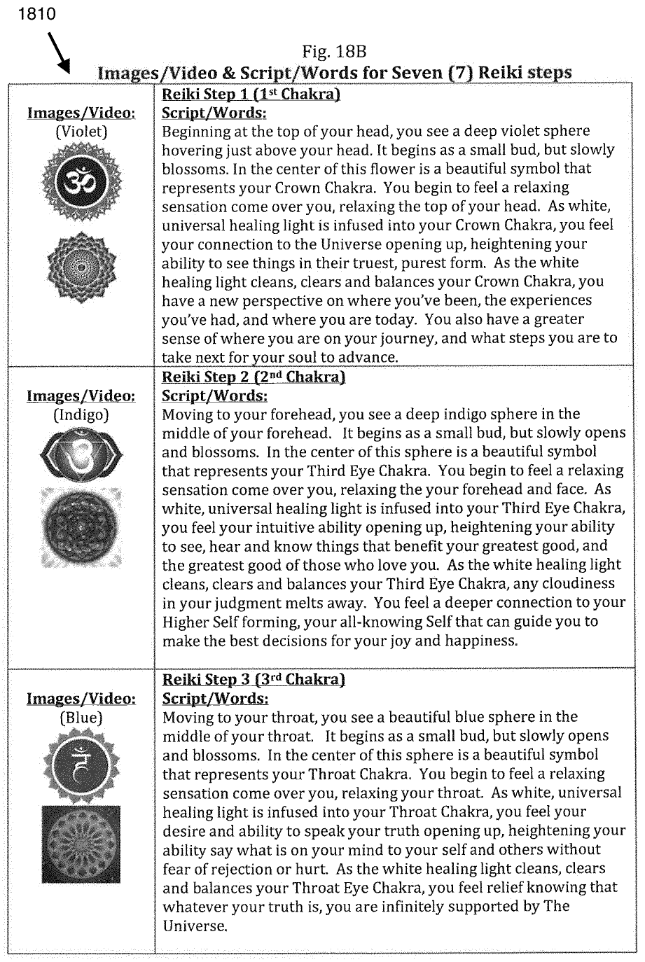

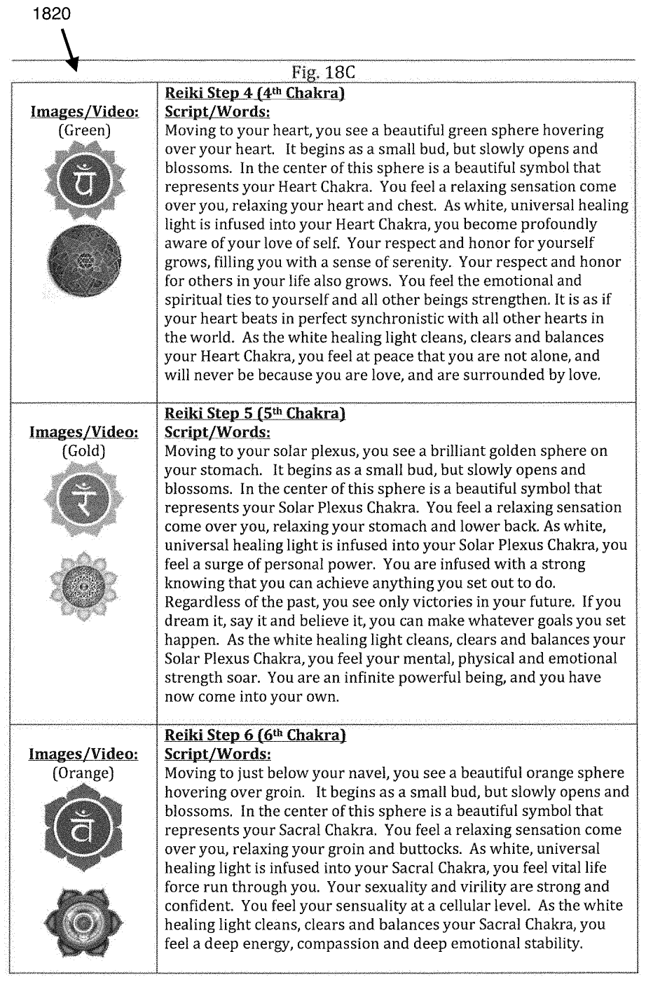

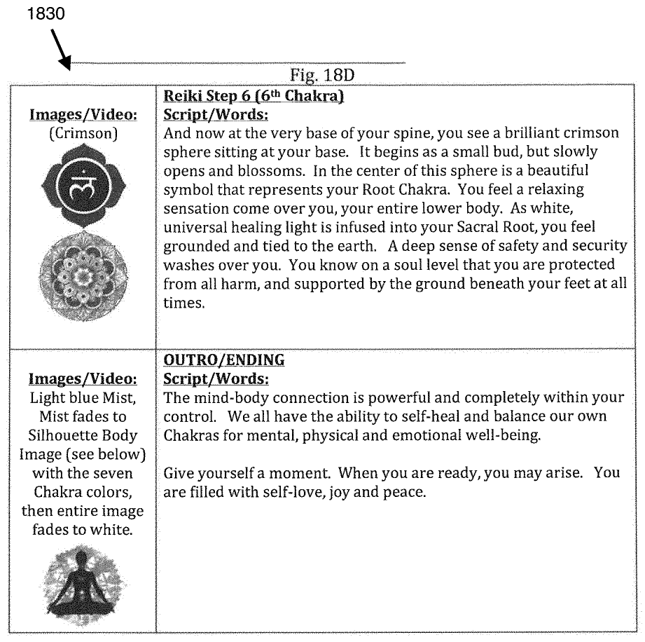

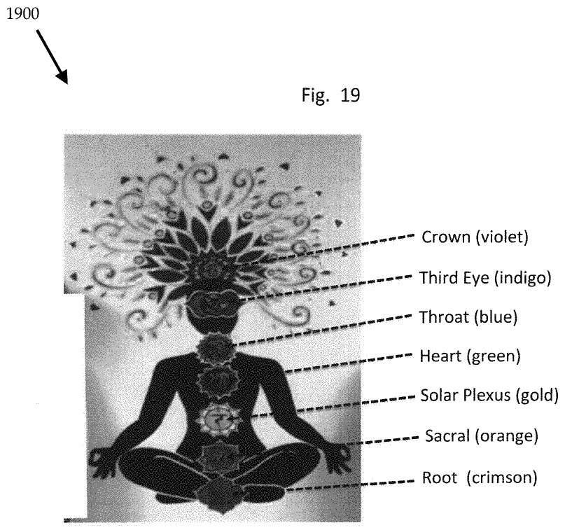

FIG. 8A is an illustration of a digital word/file data structure for audio and video files, in accordance with embodiments of the present disclosure.

FIG. 9 is a block diagram showing a multi-stage CAM treatment plan and possible adjustments thereto, in accordance with embodiments of the present disclosure.

FIG. 10A is a top-level component selection layout for Reiki steps 1-4, in accordance with embodiments of the present disclosure.

FIG. 10B is a top-level component selection layout for Reiki steps 5-7, in accordance with embodiments of the present disclosure.

FIG. 10C is a detail sub-component selection of factors/attributes for a single Reiki step where all the sensory components are present, in accordance with embodiments of the present disclosure.

FIG. 11A is a top level component selection layout for Reiki steps 1-4, with four time segments, in accordance with embodiments of the present disclosure.

FIG. 11B is a top level component selection layout for Reiki steps 5-7, with four time segments, in accordance with embodiments of the present disclosure.

FIG. 11C is a detail sub-component selection of factors/attributes for sensory components 1-2 of a single step of a Reiki treatment session having four time segments, in accordance with embodiments of the present disclosure.

FIG. 11D is a detail sub-component selection of factors/attributes for sensory components 3-5 of a single step of a Reiki treatment session having four time segments, in accordance with embodiments of the present disclosure.

FIG. 12 is a listing of various patient/client data that may be collected from a patient/client/user, in accordance with embodiments of the present disclosure.

FIG. 13 is a data-to-components top-level factors/attributes map, in accordance with embodiments of the present disclosure.

FIG. 13A is a data-to-detailed sub-component factors/attributes map for a sensory component, in accordance with embodiments of the present disclosure.

FIG. 13B, is a data-to-detailed sub-component factors/attributes map for another sensory component, in accordance with embodiments of the present disclosure.

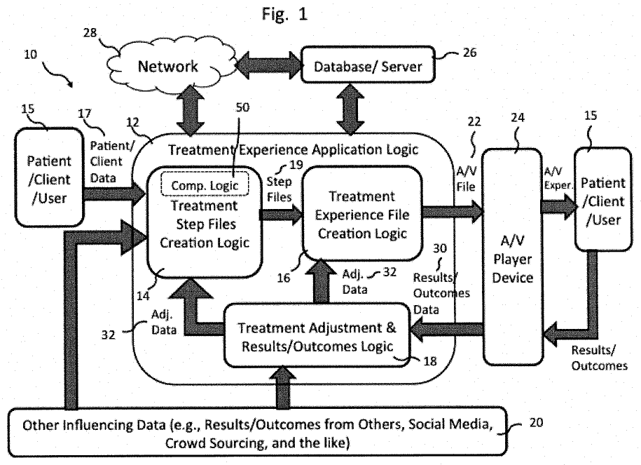

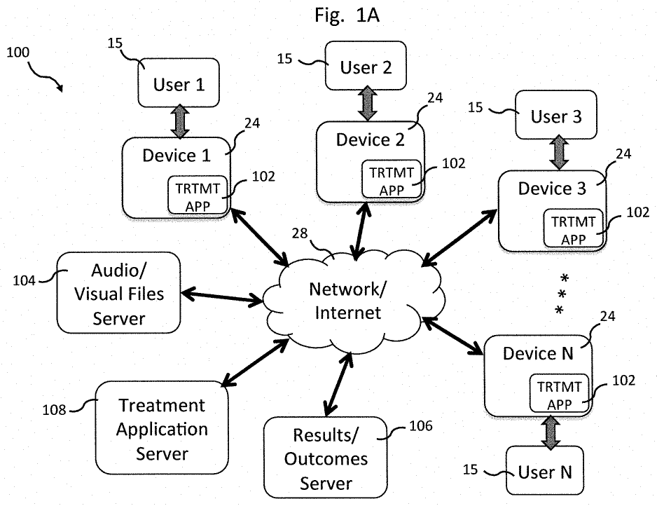

FIG. 14 is a flow diagram of one of the components in FIG. 1, in accordance with embodiments of the present disclosure.

FIG. 15 is a flow diagram of another of the components in FIG. 1, in accordance with embodiments of the present disclosure.

FIG. 16 is a flow diagram of another of the components in FIG. 1, in accordance with embodiments of the present disclosure.

FIG. 17 is an illustration of the various energy centers in the human body and default ailments associated therewith, in accordance with embodiments of the present disclosure.

FIG. 18A is a portion of a script/words text file shown and corresponding GUI images for the Reiki steps, in accordance with embodiments of the present disclosure.

FIG. 18B is another portion of a script/words text file shown and corresponding GUI images for the Reiki steps, in accordance with embodiments of the present disclosure.

FIG. 18C is another portion of a script/words text file shown and corresponding GUI images for the Reiki steps, in accordance with embodiments of the present disclosure.

FIG. 18D is another portion of a script/words text file shown and corresponding GUI images for the Reiki steps, in accordance with embodiments of the present disclosure.

FIG. 19 is as illustration of an image or graphic that may appear on a GUI as part of a treatment experience, in accordance with embodiments of the present disclosure.

### DESCRIPTION

As discussed in more detail below, methods and systems of the present disclosure provide customizable and adaptable energy healing to the patient/client/user with an audio/visual experience that allows the user to customize, accelerate, and/or optimize their own physical and emotional wellness improvement and healing from many ailments and disorders, such as such as chronic pain, obesity, addiction, and stress management.

For example, the present disclosure enables a patient with chronic or severe pain to potentially reduce or eliminate the need for pain medications, such as opiates and the like, which can be highly addictive, thereby reducing the likelihood of long term addiction or the transition from prescription pain medication to illegal street drugs, such as heroin and the like.

It is known that the mind-body connection is powerful enough to enable the body to improve physical and emotional wellness and even to heal itself from the inside out. For example, many ailments or disorders may be overcome or managed through eastern energy medicine techniques, such as Reiki. In particular, energy medicine, such as Reiki, opens-up the mind-body connection and works with the “energy centers” (or “chakras”) inside the body; however, each person responds differently to treatments and thus may require tailored or customized approaches to receive maximum benefit. It is also known that when these energy centers are blocked, people can suffer from physical and emotional ailments. Conversely, when these energy centers are cleared and balanced, people can actually improve their physical and emotional wellness and even heal themselves from the inside-out.

The present disclosure allows each patient to obtain the maximum benefit from energy medicine treatments or techniques, such as Reiki, by identifying what components work best for that person (or patient) and that particular condition being treated. The present disclosure uses digital file-based audio/video therapeutics to provide a treatment experience for patient, similar to a virtual reality experience or the like. The present disclosure also uses of analytics, “big data”, real-time global data networking, and machine learning, to obtain the latest treatment successes and failures and correlate them to patient data to optimize treatments or provide more personalized treatment regimes or plans or experiences, which is customizable, selectable and adaptable (continuously in real-time) and which adjusts and improves (continuously in real-time) the treatment experience for the current patient and other patients.

FIG. 1 illustrates various components (or devices or logic) of a computer-controlled adaptable audio/visual therapeutic treatment system 10 (or CAM treatment system) of the present disclosure, which includes Treatment Experience Application Logic 12 (or Treatment Application Logic or TRTMT App or Virtual Energy Medicine App) having various logics for performing the functions of the present disclosure including Treatment Step File Creation Logic (or Step Creation Logic) 14, Treatment Experience File Creation Logic 16 and Treatment Adjustment & Results/Outcomes Logic 18. The Treatment Application Logic 12 receives data 17 from a patient or client or user 15, indicative of the user's medical condition and various personal attributes and characteristics of the user 15. More details about the patient/client data 17 are described and shown hereinafter. The patient/client data 17 is fed to the Treatment Step Files Creation Logic 14 which determines factors and/or attributes for individual Sensory Components (discussed more hereinafter) and creates digital audio/visual (A/V) Reiki (or energy medicine) treatment step files related to each treatment step to be used in a complete energy medicine treatment session experience.

The Treatment Step Files Creation Logic 14 also receives input data from other influencing (or influential) data sources 20 (such as outcomes/results from others, social media, crowd sourcing, and/or other sources), as discussed more hereinafter. The Treatment Step Files Creation Logic 14 also receives input data from Treatment Adjustment & Results/Outcomes Logic 18 and adjusts certain factors or attributes related to creating the treatment step files in response to the data received from the Treatment Adjustment & Results/Outcomes Logic 18, as discussed more hereinafter.

The Treatment Step Files Creation Logic 14 may also have Sensory Component File Creation Logic 50 (as a portion of the overall Logic 14) which receives the patient/client data 17, other influencing (or influential) data 20 and adjustment data 32 and creates the individual Sensory Component files which may be used by another portion of the Step Creation Logic 14 to create the digital A/V Reiki step files.

The Treatment Step Files Creation Logic 14 provides digital treatment step files 19 (discussed more hereinafter) to digital Treatment Experience File Creation Logic 16, which combines a predetermined number of the treatment step files 19 in a predetermined order together with other optional treatment session packaging files, features or data, and creates a complete digital audio/visual (A/V) energy medicine treatment session experience file 22. The treatment session experience file 22 is provided to an audio/visual player device 24, which plays the digital treatment session experience file 22 for the patient or client or user 15 to experience the treatment session.

The A/V player device 24 may be any device capable of receiving and playing the A/V treatment session experience file and may be an audio-only device, such as an audio digital sound player, e.g., an iPod® or the like. Alternatively, the A/V player device 24 may be any device that provides both audio and video capability, such as any form of multi-media platform, gaming platform or virtual reality platform or headset (e.g., Samsung Gear VR®, Google Cardboard®, Occulus Rift®, HTC Vive®, Virtuix Omni™, Xbox®, OnePlus®, PlayStation VR®, Wii®, or the like), smart phone, Smart TV computer, laptop, tablet, personal e-reader, or the like. The audio data portion of the treatment session experience file may be any acceptable audio type/format, e.g., stereo, mono, surround sound, Dolby®, or any other suitable audio format, and the video portion of the treatment session experience file may be in any acceptable video type/format, such as High Definition (HD), Ultra-High Definition (UHD), 2D or 3D video, 1080p, 4K UDH (2160p) 8K UDH (4320p), 360 degrees, or any other suitable video type or format for the audio/video device playing the treatment session experience file. Any other audio/visual platform that provides the functions and performance described herein may be used if desired.

When the treatment session or experience is complete, the results or outcomes of the Reiki treatment session/experience are measured, obtained, received and/or collected from the patient/client/user 15 in the form of results/outcomes data 20, which is provided to the Treatment Adjustment & Results/Outcomes Logic 18 and the Treatment Step Files Creation Logic 14 (discussed more hereinafter). The results data 20 may be collected by the same device that delivers the audio/visual treatment experience to the user. For example, if the player device 24 is a smart phone, or other interactive device, the user 15 may be asked one or more questions after the treatment session ends and the device may record/save the responses as Results/Outcomes data 30 and provide the data to the Treatment Application Logic 12.

The Treatment Step Files Creation (TSFC) Logic 14 and Treatment Experience File Creation (TEFC) Logic 16 may also receive input data or files or commands from one or more databases or servers 26 either directly or through a network 28 to perform the functions described herein.

The Treatment Step Files Creation (TSFC) Logic 14 and Treatment Experience File Creation (TEFC) Logic 16 may also receive input data or files or commands from the Treatment Adjustment & Results/Outcomes (TARO) Logic 18. Treatment Adjustment & Results/Outcomes (TARO) Logic 18 receives input data from the Other Influencing Data (discussed herein) 20 and Results/Outcomes data 30 from the A/V Player Device (or A/V Device) 24 and provides treatment adjustment data 32 to the TSFC or TEFC logics, respectively, which determine whether the TSFC Logic 14 or the TEFC Logic 16 needs adjustment to improve or optimize the treatment results/outcomes, or it may directly modify certain databases or servers 26 to adjust the files accessed by, or the results provided by, the TSFC Logic 14 or TEFC Logic 16.

Referring to FIG. 1A, a network block diagram 100 of various components of an embodiment of the computer-controlled adaptable treatment system of the present disclosure, includes a plurality of computer-based A/V devices (Device 1 to Device N) which may interact with each other and with respective users (User 1 to User N) (or patients or clients) each user being associated with one of the devices. Each of the computer-based devices 24 may include a respective local (or host) operating system running on the computers of the respective devices 24. Each of the devices 24 includes a respective audio playing interface and audio drivers for playing an audio file and may also include a display screen that interacts with the operating system and any hardware or software applications, video drivers, interfaces, and the like, needed to play the desired audio content and display the desired visual content on the respective display. The users 15 interact with the respective devices 24 and may provide input data content to the devices 24 using the displays of the respective devices 24 (or other techniques) as described herein.

Each of the computer-based A/V devices may also include a local Treatment Experience application software 102 (or “Treatment App” or “TRTMT App” or “TE App”), running on, and interacting with, the respective operating system of the device 24, which may receive inputs from the users 15, and provides audio and video content to the respective speakers/headphones and displays of the devices. In some embodiments, the Treatment App 102 may reside on a remote server and communicate with the A/V device via the network 28.

The A/V devices 1-N may be connected to or communicate with each other through the communications network 28, such as a local area network (LAN), wide area network (WAN), virtual private network (VPN), peer-to-peer network, or the internet, by sending and receiving digital data over the communications network. If the devices are connected via a local or private or secured network, the devices may have a separate network connection to the internet for use by the device web browsers. The devices 24 may also each have a web browser to connect to or communicate with the internet to obtain desired content in a standard client-server based configuration, such as YouTube® or other audio/visual files, to obtain the Treatment App 102 and/or other needed files to execute the logic of the present disclosure. The devices 24 may also have local digital storage located in the device itself (or connected directly thereto, such as an external USB connected hard drive, thumb drive or the like) for storing data, images, audio/video, documents, and the like, which may be accessed by the Treatment App running on the A/V device.

In addition, the computer-based A/V devices 24 may also communicate with a separate audio/video content computer server 104 via the network 28. The audio/video content server 104 may store the audio/video files (e.g., sensory component files, audio/visual experience files, audio or visual selection files, libraries, or databases, and the like) described herein or other content stored on the server desired to be used by the devices 24. The devices 24 may also communicate with a results/outcomes computer server 106 via the network 28, which may store the results/outcomes data from all the users 15 of the Treatment App 102. The devices 24 may also communicate with a Treatment Application computer server 108 via the network 28, which may store the latest version of the Treatment Application software 102 (and may also store user attributes and settings files for the Treatment App, and the like) for use by the users of the devices 1-N to run (or access) the Treatment App 102. These servers 104-108 may be any type of computer server with the necessary software or hardware (including storage capability) for performing the functions described herein. Also, the servers 104-108 (or the functions performed thereby) may be located in a separate server on the network 28, or may be located, in whole or in part, within one (or more) of the devices 1-N on the network 28.

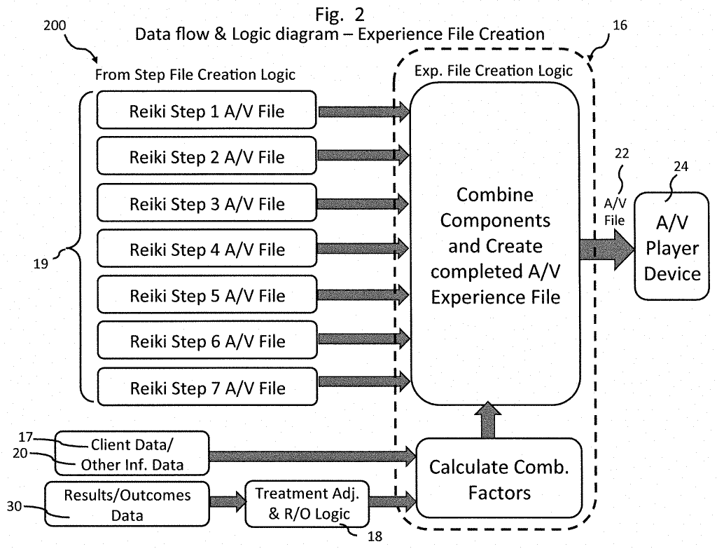

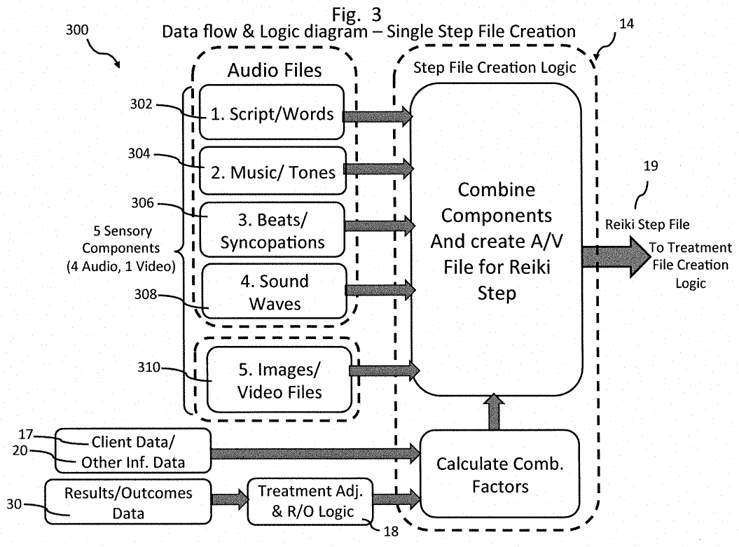

Referring to FIG. 2, a data flow diagram 200 shows the treatment step files 202 created by the Step Files Creation Logic 14 (FIG. 1) that are provided to the Treatment Experience File Creation Logic 16, which receives the patient/client data 17, the other influential data 20, and the treatment adjustment data from the Treatment and Adjustment Logic 18 and uses the data to select, combine, and adjust (as needed) specific audio files and visual files from the digital treatment (or Reiki) step files 19 in a predetermined order (as discussed hereinafter) together with other optional treatment session packaging files, features or data, and creates the digital audio/visual (A/V) treatment session experience file 22.

Referring to FIG. 3, a data flow diagram 300 shows the Treatment Step Files Creation (TSFC) Logic 14 (FIG. 1) which receives the patient/client data 17, the other influential data 20, and the treatment adjustment data from the Treatment and Adjustment Logic 18 and uses the data to select, combine, and adjust (as needed) specific audio files and visual files from several “Sensory Components” files to create each digital treatment step file. The Treatment Step Files Creation Logic 14 provides the treatment step files 19 for each of the Reiki (or treatment) steps to be performed/delivered to the patient/client/user 15.

In particular, there may be five (5) sensory components files 302-310, comprising four (4) audio files 302-308 and one (1) video file 310, all of which may be combined in a predetermined way to create each digital treatment step file 19. The four (4) audio files 302-308 may be, e.g., script/words, music/tones, beats/syncopation (or binaural beats), and sound-wave therapy (or SWT or Sound Waves), and the video file 310, may be, e.g., images/video. In particular, the script/words audio file 302 (Sensory Component 1) may be the scripted voice that is spoken to the patient/user 15 during the audio/visual treatment session experience. It may consist of a specific scripted spoken text or message made to obtain a desired experience or response from the user's body. The music/tones (or music/tones/sounds) audio file 304 (Sensory Component 2) may be a composition of music, tones and/or other types of sounds (e.g., nature sounds), made to obtain a desired experience or response from the user's body.

The binaural beats/syncopation file 306 (Sensory Component 3) may be an audio file that simultaneously provides a marginally different sound frequency (or tone) to each ear through headphones. Upon hearing the two tones, the brain interprets the tones sent to the left and right ears as one tone. The interpreted single tone is equal in measurement (Hertz) to the difference between the source tones. For example, if a 205 Hz sound frequency is sent to the left ear, and a 210 Hz sound frequency is sent to the right ear, the brain will process and interpret the two sounds as one 5 Hz frequency. The brain then follows along at the new frequency (5 Hz), producing brainwaves at the same rate (Hz). This is also known as the “frequency following response.”

Binaural beats recreate brainwave states, and are able to bring the brain to different states, of which there are four (4) categories (or states):
(i) Beta (14-40 Hz) associated with concentration, arousal, alertness, cognition (higher levels associated with anxiety, disease, feelings of separation, fight, or flight);
(ii) Alpha (8-14 Hz) associated with relaxation, super-learning, relaxed focus, light trance, increased serotonin production, pre-sleep, pre-waking drowsiness, meditation, beginning of access to unconscious mind;
(iii) Theta (4-8 Hz) associated with dreaming sleep (REM sleep), increased production of catecholamine (related to learning and memory), increased creativity, integrative, emotional experiences, potential change in behavior, increased retention of learned material, hypnogogic imagery, trance, deep meditation, access to unconscious mind; and
(iv) Delta (1-4 Hz), associated with dreamless sleep, human growth hormone released, deep, trance-like, non-physical state, loss of body awareness, access to unconscious and “collective unconscious” mind.

The Sound Waves or sound frequency therapy file 308 (Sensory Component 4) is an audio file that provides sound waves at audio frequencies which may be audible or inaudible to the human ear, but which provide therapeutic, relaxation or healing effects.

The audio frequencies may be stationary or swept across a predetermined frequency range at a given rate, with a given amplitude profile to optimize the effects of this sensory component. Any type of sound waves or audio frequencies and frequency ranges may be used if desired for Sensory Component 4, generally referred to herein as Sound Waves, depending on the type of disease or disorder being treated to obtain a desired experience or response from the body.

The Images/Video file 310 (Sensory Component 5) is a visual file that provides still images or videos (or moving images), having a specific length, which is made to obtain a desired experience or response from the user's body.

Other audio and video files may be used if desired. Also, other types and numbers of sensory components and sensory component files may be used if desired. Also, some of the sensory components may be combined into one sensory component or split-up to create more sensory components, if desired.

Referring to FIGS. 4-8, for each of the Sensory Components (1-5) there may be corresponding separate Sensory Component File Creation Logics 401,501,601,701,801, or a single Sensory Component File Creation Logic (referred to collectively as 50 (FIG. 1), which may be a portion of the Step Creation Logic 14 (FIG. 1), receives the patient/client data 17, the other influential data 20, and the treatment adjustment data from the Treatment and Adjustment Logic 18 and uses the data to create the Sensory Component Files, which are provided to the Step Creation Logic 14 (or portion thereof). Also, each sensory component file(s) may be made up of several sub-components associated with that sensory component, as discussed hereinafter.

In particular, referring to FIG. 4, a data flow and component block diagram 400 shows Sensory Component 1 Creation Logic 401, which creates the Sensory Component 1 (Script/Words) File 302, and may have six (6) sub-components, e.g., script text and length 402, languages 404, voice type 406, narration style 408, speed 410 and volume/special effects 412. Other types and numbers of sub-components may be used for any of the Sensory Components 302-310, if desired.

Referring to FIG. 5, a data flow and component block diagram 500 shows Sensory Component 2 Creation Logic 501, which creates the Sensory Component 2 (Music/Tones or Music/Tones/Sounds) File 304 (FIG. 3), and may have six (6) sub-components, e.g., musical score and length 502, musical keys 504, instrument/tone/sound types 506, voice type 508, rhythm/cadence/speed 510 and volume/special effects 512. This sensory component may also include sounds in nature, such as the sounds of the ocean, animals (e.g., birds chirping, dogs barking, cats purring/meowing, and the like), or machines/man-made sounds (e.g., traffic, clock ticking, footsteps, phone ringtones, computer tones, cars, motorcycles, mechanical machinery, and the like) or any other sound. Multiple instrument/tone/sound types may be used in a given segment, e.g., singing voice with flute music and with ocean sound in the background.

Referring to FIG. 6, a data flow and component block diagram 600 shows Sensory Component 3 Creation Logic 601, which creates the Sensory Component 3 (Beats/Syncopation) File 306 (FIG. 3), and may have six (6) sub-components, e.g., beats segment & length 602, musical keys 604, instrument/tone types 606, voice type 608, rhythm/cadence/speed 610 and volume/special effects 612.

Referring to FIG. 7, a data flow and component block diagram 700 shows Sensory Component 4 Creation Logic 701, which creates the Sensory Component 4 (Sound Waves) File 308 (FIG. 3), and may have three (3) sub-components, e.g., frequency range and segment time length 702, speed (e.g., sweep rate or repetition rate) 704 and amplitude/special effects 706.

Referring to FIG. 8, a data flow and component block diagram 800 shows Sensory Component 5 Creation Logic 801, which creates the Sensory Component 5 (Images/Video) File 310 (FIG. 3), and may have three (3) sub-components, e.g., images 802, video and length 804, and brightness/special effects 806.

Other types and numbers of sub-components may be used for any of Sensory Components 1-5, if desired.

FIGS. 4A, 5A, 6A, 7A, and 8A, show digital word/file data structures for audio and video files that may be used with embodiments of the present disclosure.

Referring to FIG. 4A, in particular, an illustration 450 of digital word/file data structures shows options for creating various audio files for the script/word Sensory Component 1 file, which show three (3) groupings, one group 452 for 250 word scripts, another group 454 for 500 word scripts, and a third group 456 for 750 word scripts. Each script length may be recorded and saved as a digital file having one or more of the attributes/sub-components, such as a voice type of Male, Female, or Child, and having a Narration Style 1 to n, spoken in a language 1 to n, at a speed 1 to n. Alternatively, the script/word files may be grouped by time duration or length (e.g., seconds, minutes, or hours) of the script/words segment (e.g., 5 min., 10 min., 15 min.). These files may be repeated for as many combinations of the attributes/sub-components as desired. The files may be stored in a library or database or server (local or via a network), e.g., in the audio/visual files server 104 (FIG. 1A), that can be selected and accessed by the corresponding Sensory Component File Creation Logic 401 (FIG. 4). After one or more of the audio script/words files are determined or selected with the desired attributes (based on the input data), the volume and special effects may be added to create the (script/words) Sensory Component 1 file 302 that is sent to or accessed by the Step File Creation Logic 14 (FIG. 3).

Referring to FIG. 5A, an illustration 550 of digital audio data file structures shows options for creating various audio files for the music/tones Sensory Component 2 files, which may have three (3) groupings, one group 552 for 250 notes musical score, another group 554 for 500 note score, and the third group 556 for 750 note score. Each musical score length is recorded and stored having one (or more) of the attributes/sub-components, such as a musical key 1 to n, instrument/tone 1 to n, voice type of Male, Female, or Child, and pitch/tone (e.g., alto, soprano, tenor, base, or other) and at a word speed 1 to n (e.g., how quickly the words are spoken and the duration of spaces between words). Alternatively, the musical score/segments files may be grouped by time duration or length (e.g., seconds, minutes, hours) of the musical score/segment (e.g., 5 min., 10 min., 15 min.). These files may be repeated for as many combinations of the attributes/sub-components as desired. The files may be stored in a library or database or server (local or via a network), e.g., in the audio/visual files server 104 (FIG. 1A), that can be selected by the corresponding Sensory Component File Creation Logic 501 (FIG. 5). After one or more of the audio music/tones files are determined or selected with the desired attributes, the volume and special effects can be added to create the Sensory Component 2 (music/tones) file 304 that is sent to or accessed by the Step Creation Logic 14. Other segment lengths or groupings may be used if desired.

Referring to FIG. 6A, an illustration 650 of digital audio data file structures shows options for creating various audio files for the binaural beats/syncopation Sensory Component 3 files 306 (FIG. 3), which may have three (3) groupings based on segment time duration or length, one group 652 for 5 min. beat segment, another group 654 for 10 min. beat segment, and the third group 656 for 15 min. beat segment. Each beat segment length may be recorded having one of the attributes/sub-components, such as a musical key 1 to n, instrument/tone 1 to n, voice type of Male, Female, or Child, and pitch/tone (e.g., alto, soprano, tenor, base or other) and at a speed 1 to n. Alternatively, the binaural beat segments may be grouped by binaural beat frequency (or following frequency) (e.g., 5 Hz, 10 Hz, 15 Hz) of the beat segment or the frequencies provided to each ear, (e.g., 210 Hz/200 Hz, 350 Hz/340 Hz, 110 Hz/100 Hz). The files may be stored in a library or database or server (local or via a network), e.g., in the audio/visual files server 104 (FIG. 1A), which can be selected by the Sensory Component File Creation Logic. After one or more of the audio binaural beats files are determined or selected with the desired attributes, the volume and special effects can be added to create the Sensory Component 3 (binaural beats/syncopation) file 306 that is sent to or accessed by the Step Creation Logic 14. Other segment lengths or groupings may be used if desired. Also, other beat frequency or syncopation techniques may be used for the Sensory Component 3 to create desired brain wave states.

Referring to FIG. 7A, an illustration 750 of digital audio data file structures shows options for creating various audio files for the sound-wave Sensory Component 4 files 308 (FIG. 3), where each Sound Wave segment length is recorded having a given combination of the attributes/sub-components, such as a particular frequency range, sweep rate, repeat rate, and the like (referred to simply as Sound Wave or SW 1-N) for different durations or lengths of time the segment lasts. For the sound wave, various sound wave segments may have three (3) groupings based on segment time duration or length, one group 752 for a 5 minute sound wave segment, another group 754 for 10 minute sound wave segment, and the third group 756 for 15 minute sound wave segment. These files may be repeated for as many combinations of the attributes/sub-components as desired. The files may be stored in a library or database or server (local or via a network), e.g., in the audio/visual files server 104 (FIG. 1A), that can be selected by the Sensory Component File Creation Logic. After one or more of the audio sound-wave files are selected with the desired attributes, the amplitude and special effects may be added to create the Sensory Component 4 (sound-wave or sound wave) file 308 that is sent to or accessed by the Step Creation Logic 14. Other segment lengths or groupings may be used if desired.

Referring to FIG. 8A, an illustration 850 of digital video/image data file structures shows options for creating the various images/video files for the images/video Sensory Component 5 files 310 (FIG. 3) are shown, where there may be two visual file formats: images 852 and videos 854. For images 852, there may be a library or database of images in a database or server (local or via a network), e.g., in the audio/visual files server 104 (FIG. 1A) that can be selected by the corresponding Sensory Component File Creation Logic 801. After one or more image files are selected, the brightness and special effects may be added to achieve the desired visual effect. For the video segments 854, various video segments may have three (3) groupings based on segment time duration or length, one group 856 for a 5 minute video segment, another group 858 for 10 minute video segment, and the third group 860 for 15 minute video segment. Each video segment length may be recorded and saved having a given combination of the attributes/sub-components, such as a musical key 1 to n, instrument/tone 1 to n, voice type of Male, Female, or Child, and tone (alto, soprano, tenor, base) and at a speed 1 to n. These files may be repeated for as many combinations of the attributes/sub-components as desired. The files may be stored in a library or database or server (local or via a network), e.g., in the audio/visual files server 104 (FIG. 1A), that can be selected by the corresponding Sensory Component File Creation Logic 801 (FIG. 8). After one or more of the video files are determined or selected with the desired attributes (based on the input data), the brightness and special effects may be added to create the Sensory Component 5 (images/video) file 310 that is sent to or accessed by the Step Creation Logic 14 (FIG. 3). Other segment lengths or groupings may be used if desired.

Referring to FIG. 9, a block diagram 900 shows the treatment adjustment & results/outcomes logic 18 and how it may relate to a multi-stage CAM treatment plan and possible adjustments thereto. In particular, the outcomes/results data 30 (FIG. 1) obtained from patients/clients/users 15 are assessed by the present system to identify whether a given CAM treatment program or plan having multiple CAM stages should be adjusted to optimize treatment results for a given patient/client/user. For example, additional treatments may be added if the results from this patient or other patients with similar conditions and other applicable attributes have benefited from such a change. The present system may be constantly learning from the results/outcome data 30 to improve or optimize a given treatment regimen, shown as CAM Treatments 1-N in FIG. 9. Such learning or optimization may be done by known machine learning, expert systems, predictive analytics/modeling, pattern recognition, mathematical optimization, learning algorithms, neural networks or any other techniques and technology that enable the treatment experience A/V files provided to the patient/client/user to improve the results/outcomes over time. In particular, the logic 18 may receive positive and negative results data from users, and use that data to train the logic 18 to identify what parameters work best for users with certain input characteristics. Such correlations, or predictions, or classifications may be learned over time by the logic of the present disclosure, using machine learning techniques and classifiers, such as support vector machines (SVMs), neural networks, decision tree classifiers, logistic regression, random forest, or any other machine learning or classification techniques that perform the functions of the present disclosure. This would also apply for the composition of a given single treatment session, and the make-up and number of the Sensory Components.

FIGS. 10A and 10B show a top-level component selection layout for seven Reiki steps, in accordance with embodiments of the present invention. Referring to FIGS. 10A and 10B, a top level layout 1000 for Reiki steps 1-4 (FIG. 10A) 1006-1010 and a top level layout 1050 for Reiki steps 5-7 (FIG. 10B) 1012-1014 are shown with the Sensory Components in a left column 1002 (each Reiki step having 5 possible sensory components, as discussed herein) and the top level selection in a right column 1004 showing whether or not a given component has been selected to be in each Reiki step. If the selection in column 1004 shows “(none),” then that sensory component is not included in that Reiki step. In particular, for example, for Reiki step 1 layout 1006, all the Sensory Components 1-5 are included in that step. Further, for example, for Reiki step 2 layout 1008, the Binaural Beats/Syncopation and Sound Waves Sensory Components are not included in that step (as indicated by the “none” in those fields); however, the remaining Sensory Components are all present. The remaining Reiki steps 3-7 are self explanatory from the FIGS. 10A and 10B.

Referring to FIG. 10C, a detail layout 1080 showing sub-component selection of factors/attributes for a single step of a Reiki treatment session where all the sensory components are present, such as in Reiki step 1 of FIG. 10A, is shown. In particular, the combination of all the sub-components shown in this example, may be a Reiki (treatment) Step 1 file provided by the Step Creation Logic 14. The selected factors/attributes (sub-components) are shown for those selected, and for those not present, it shows “None” in the factors/attributes column. In particular, for the Script/Words Component file 302, there is a specific script of 250 words (Code S1250), in English, with a Male voice, a UK accent, having a speed of 5, a volume of 5, and an echo special effect on the voice. For the Music/Tones Component file 304, there is a musical score having 750 notes, in the key of A sharp, played on ceramic crystal, with No voice, having a speed of 5, a volume of 4, and no special effects. The remaining sensory component files 306-310 in FIG. 10C operate in a similar way, which should be understood in view of the discussion herein.

Referring to FIGS. 11A and 11B, a top level component selection layouts 1100 and 1150, respectively, are shown for Reiki steps 1-4 (FIG. 11A) and Reiki steps 5-7 (FIG. 11B), with four (4) time segments (Segment1, Segement2, Segement3, Segment4) for each Reiki step and each Sensory Component. If the selection shows a blank, then that component is not included in that time segment. In particular, for Reiki step 1 layout, all the sensory components are included in the first time segment (Segment 1); only the Music/Tones and Images are included in Segment 2; only the Script/Words, Sound Wave, and Video are included in Segment 3; and all the components except for the Beats/Syncopation are included in Segment 4. Having multiple time segments in a given Reiki step provides the flexibility to have multiple different combinations of audio and/or visual experience in a given Reiki step. A similar approach is followed for Reiki steps 2-7 in FIGS. 11A and 11B.

Referring to FIGS. 11C and 11D, detailed layouts 1180 and 1190, respectively, are shown having a detail sub-component selection of factors/attributes for components 1-2 (FIG. 11C) and components 3-5 (FIG. 11D) of a single step of a Reiki treatment session having four time segments, where all components are present, such as in Reiki step 1 of FIG. 11A. Referring to FIGS. 11C and 11D, the combination of all the sub-components shown in this example, may be a Reiki (treatment) Step 1 file provided by the Step Creation Logic. The selected factors/attributes for the sub-components are shown for those selected, and for those not selected it shows “None” in the factors/attributes column. In particular, for the Script/Words Component file 302, there is a specific script of 250 words, in English, with a Male voice, a UK accent, having a speed of 5, a volume of 5, and an echo special effect on the voice. For the Music/Tones Component file 304, there is a musical score having 750 notes, in the key of G, played on the flute, with No voice, having a speed of 5, a volume of 4, and no special effects. The remaining sensory component files 306-310 in FIGS. 11C and 11D operate in a similar way, which should be understood in view of the discussion herein.

FIG. 12 shows a listing 1200 of various patient/client/user data 17 (FIG. 1) that may be collected from the patient or client or user 15 of the system 10 of the present disclosure. The data 17 is segmented into groups or categories, such as “Hard” Facts (e.g., attributes or characteristics that do not change about a person), “Soft” Facts (e.g, attributes or characteristics that that may be subjective or based on testing data), Medical Condition (e.g., includes what the patient is currently requesting treatment for), Current Traditional Medical Treatment (e.g., what types of traditional medical treatment is the patient currently undergoing), Current CAM Medical Treatment (e.g., what type of CAM treatment is the patient currently undergoing), Environment (e.g., where is the patient from currently, and what time of day, date, and day of week is it), Requirements/Desired Outcome(s) (e.g., are there any time constraints on treatment, and what is the desired outcome of treatment), Other Influencers (e.g., any other influencing factors not covered by the other categories or groupings, such as social media activity or use, general territory information, other patient results/outcomes, and the like). More or less or different data may be used if desired. The patient/client data 15 (FIG. 1) may be used along with other data to determine the appropriate factors and/or attributes for each Sensory Component to create each Reiki treatment step and to create the complete Reiki treatment session experience file, as described herein.

FIG. 13 is a data-to-components top-level factors/attributes map 1300 showing how given patient/client data 15 (e.g., like that shown in FIG. 12) may be mapped (at a top-level) to whether or not a given sensory component will be used in a given Reiki step. In particular, in FIG. 13, for a male (first item in “Hard” Facts), Reiki step 1 would include Script/Words, Music/Tones, Sound Waves and Images/Video components, but not include the Beats/Sync component; and Reiki step 2 would include Music/Tones, Sound Waves and Images/Video components, but not include Script/Words nor Beats/Sync components. This table may have values preset as a default parameters, and/or may be learned and updated over time, such as by the update logic 18 (FIG. 1), using machine learning or the like as discussed herein.

Referring to FIGS. 13A and 13B, data-to-detailed sub-component factors/attributes maps 1350 and 1380, respectively, are shown for Component 1 (FIG. 13A) and Component 2 (FIG. 13B), showing how given patient/client data may be mapped to the detailed sub-component factors to be used in a given Reiki step (e.g., Reiki step 1 in FIG. 13). In particular, in FIG. 13A, for a male (first item in “Hard” Facts), Sensory Component 1 (Script/Words) for Reiki step 1, would include Script S1250 (Script#1, 250 words), in English, with a Male voice, having a UK accent, at a speed of 5, and volume of 5, and an echo special effect. Also, for Age range 2, as there was no Script/Words component for Reiki step 1 for Age range 2 in the top level map of FIG. 13, the corresponding row in the detailed factors of FIG. 13A shows “n/a” for all entries for the Scripts/Words Sensory Component 1. The remaining rows in the map in FIG. 13A operate in a similar way, which should be understood in view of the discussion herein.

Referring to FIG. 13B, for a male, Sensory Component 2 (Music/Tones) for Reiki step 1, would include musical score M3750 (Score#3, 750 notes), in key of A sharp, on a ceramic crystal, with no voice, a speed of 5 and a volume of 4, with no special effects. Also, for Gender-Female, as there was no Music/Tones component for Reiki step 1 for Gender-Female in the top level map of FIG. 13, the corresponding row in the detailed factors of FIG. 13B shows “n/a” for all entries for the Music/Tones Sensory Component 2. The remaining rows in the map in FIG. 13B operate in a similar way, which should be understood in view of the discussion herein.

FIGS. 13, 13A and 13B are two-dimensional maps indicating component and sub-component factors for a selected set of patient/client data. It should be understood that a multi-dimensional map or matrix or table or database may be created which maps (or correlates) each combination of patient/client data collected to the respective Sensory Components and sub-components. For example, there may be a mapping line item that indicates a specific set of sensory components factors for a patient/client of: male, age 26-50, weight 150-250 lbs, having personality type 1, with Lung Cancer, undergoing chemotherapy treatment plan 1. Alternatively, there may be a priority order or scaling effect of the map, such that a baseline treatment map is generated for given gender, age range, weight range, and disease state, and the other input data may cause only slight adjustments (low weighting factors) to the baseline treatment plan. Any other mapping or algorithmic approach that determines, calculates, correlates, or maps the factors/attributes of sensory components for an audio/visual treatment experience file using patient/client data and outcomes/results data and other influencing data, may be used if desired.

In some embodiments, the Component File Creation Logic 50 (FIG. 1, generally), or specifically the logics 401-801 (FIGS. 4-8), may perform a correlation or cross-correlation of the results/outcomes data for a given treatment used with one or more other patients/clients (having similar patient/client data to the current patient/client) against the patient/client data for the current patient/client, and identify the most desirable sub-components for each of the Sensory Components 1-5, and the most desirable order and number of treatment steps, to provide a desired set of sub-component factors/attributes. In some embodiments, the logic 50 may use a weighted selection (or factors) process of each of the sub-component options to determine which set would be most likely to provide the best outcomes for the current patient/client. The logic can then obtain the A/V files corresponding most closely to the desired set of attributes to create the treatment experience file for delivery to the A/V Device for the current patient/client.

The term “code” used in FIGS. 10C, 11C, 11D, 13A and 13B, is used herein as a pointer or file name or tag or label for a particular audio or video selection (or portion thereof) having a given combination of certain sub-components, that may be stored in a database, e.g., in the audio/visual files server 104 (FIG. 1A), and may have a digital file data format such as that shown in FIGS. 4A-8A. For example, a Code of S1150 may be a tag for an audio file with Script#1 having 150 words. There may be additional or alternative tags for audio or visual files having all or a set number of sub-components.

Referring to FIG. 14, a flow diagram 1400 illustrates one embodiment of a process or logic for implementing the Treatment Application Logic (or Treatment Experience Application Logic) 12 (FIG. 1). The process 1400 begins at a block 1402 which receives patient/client data 17 (FIG. 1). Next a box 1404 determines whether there is any result/outcomes data or other influential (or influencing) data available. If YES, the a block 1406 obtains the results/outcomes and other influential data and adjusts the factors/attributes/combinations model (as needed). Next, or if there is no results/outcomes and other influential data available, a block 1408 determines factors/attributes for each sensory component based on the patient/client data and creates the Reiki step files (as discussed hereinbefore) for the target A/V player device 24 (FIG. 1). If the player device 24 only plays audio or if only audio files are available, then the image/video sensory component (Sensory Component 5) may not be included in the file creation, or it may be included in the file and ignored by the A/V device 24. Also, the factors/attributes are determined for a single treatment or for a multi-stage treatment plan, such as that shown in FIG. 9. Next, a block 1410 combines the Reiki step files in a selected order and inserts any desired transition segments. For example, for certain types of medical conditions or disorders, there may only be 3 Reiki steps (e.g., steps 1, 3 and 6). Also, for another condition, there may be 7 Reiki steps but not done in sequential numerical order (e.g., steps 2, 3, 5, 1, 7, 6, and 4). Further, there may be audio/visual transition segments that are placed at the beginning or end of any given Reiki step, such as an introduction (or INTRO) to the first Reiki step 1, or an “outro” (or exit transition) after the final Reiki step 7, or there may be a desired transition between certain steps to prepare the listener to the transition. Next, a block 1412 creates the audio/visual digital treatment session experience file and provides it to the player A/V device 24. Alternatively, the logic may store the A/V experience file on a file server, e.g., the Treatment Application Server 108 (FIG. 1A), which may be accessible by the player device 24 via the computer network 28, such as the internet.

Referring to FIG. 15, a flow diagram 1500 illustrates one embodiment of a process or logic for implementing the results/outcomes portion of the Treatment & Results/Outcome Logic 18 (FIG. 1). The process 1500 begins at a block 1502, which determines whether short-term results/outcomes are available. If YES, a block 1504 receives current treatment results/outcome data from the online patient/client assessment or from another source. Next, or if there is no short-term results/outcomes data, a block 1506 determines whether there is any long term results/outcomes data. If YES, a block 1508 receives the long term results/outcomes data from various sources, including patient assessment, doctor assessment, hospital admission/discharge/re-admission data, insurance claim data, drug/pain medication prescription data, measurement data (e.g., temperature sensing, pain sensing, vital signs, ultrasound/xray, etc). Next, or if there was no long term data, a block 1510 determines whether the result/outcomes data was objectively verified. If NO, the logic adjusts the results/outcomes data to account for the subjectivity or non-obj ective measures. Next, or if the results were objectively verified, a block 1514 adjusts the results/outcomes data for redundant or conflicting data. Next, a block 1516 provides the adjusted results/outcomes data, which may be used by the Treatment Adjustment & Results/Outcome Logic 18.

Referring to FIG. 16, a flow diagram 1600 illustrates one embodiment of a process or logic for implementing the treatment adjustment portion of the Treatment & Results/Outcome Logic 18 (FIG. 1). The process 1600 begins at a block 1602, which receives results/outcomes data 32 from the user 15. Next, a block 1604 determines whether there result/outcomes data is positive, i.e., whether the current treatment A/V files are providing the desired results. If NO, the treatment experience is adjusted and a block 1606 determines which factors/attributes/combinations of which sensory components and sub-components need to be changed in the digital files to improve the results (as discussed herein). This may be done for a single treatment session, or a multi-stage treatment plan such as that shown in FIG. 9. Next, a block 1608 makes changes to the factors/attributes/combinations of the selected sensory components and sub-components in the digital files. Next, or if there was positive result/outcomes, a block 1610 receives other influencing (or influential) data. Next, a block 1612 determines whether the other influential data indicates results/outcomes (positive or negative) for a similar patient/client data to the present patient/client being treated. Other influencing data may be data from global social media, crowd sourcing, and the like that may be analyzed for trending information or other information relating to treatment effectiveness or new treatment approaches that might influence how certain treatments should be performed or adjusted. The logic 1600 may also look at global results trends data through social media for certain common traits and flag them for immediate use or immediate discontinued use. For example, if separate patients/clients in Europe, China and India have tried a unique new set of tones or music that had particularly fast results, such information may be distributed to other users and incorporated (after verification) into a patient/client treatment with a similar condition and personal attributes in the US.

If the result of block 1610 is NO, no other influential data is available for a similar patient/condition, and the process exits. If YES, influencing data is available and a block 1614 determines which factors/attributes/combinations of which sensory components and sub-components to change in the digital experience files to improve the results/outcomes based on the other influential data. This may be done for a single treatment session or a multi-stage treatment plan such as that shown in FIG. 9. Next a block 1616 makes changes to the factors/attributes/combinations of the selected sensory components and subcomponents and the logic exits. Such updates to digital treatment files may occur in real-time as global data and user analytics from other patients/clients/users is received (over internet or other network) and verified. In some embodiments, the blocks 1604 and 1612 may just receive other results/outcomes data and influential data, respectively, whether or not it is positive or for a similar patient/client data, so this data case be used to update other aspects of the treatment experience, for use on other patients or future patients. Other techniques for handling of other influential data or results/outcomes data may be used if desired, and may depend on verifiability of the data/results.

Referring to FIG. 17, an illustration 1700 of a human body 1703 and corresponding table 1701 showing the various energy centers (column 1702) in the human body 1701 and default physical ailments (column 1704) and emotional ailments (column 1706) currently known in energy medicine to be associated with each of the energy centers, as well as the colors associated with each energy center. The table 1700 may be viewed as a default table stored in a server or database, e.g., the Treatment Application Server 108, for use by the systems and methods of the present disclosure, and may be updated by the system 10 as the system learns which energy areas are most effective for certain type of patients with certain types of illnesses or disorders.

The Sensory Components may be viewed as “layers” that make up the treatment session experience file. Also, each Reiki step may be referred to as a “chakra” or energy center. An example of an embodiment of the Sensory Components (or layers) of a given treatment session experience file is shown below:
1) Script/Word—Sensory Component 1. A voiceover script describing the experience, e.g., approximately 3 minutes per chakra (or Reiki step or energy center) for a total treatment session length of, e.g., 21 minutes. Other time lengths may be used if desired.
2) Music/Tones—Sensory Component 2. Original musical composition that may modulate across seven (7) musical keys, each key resonating with a specific energy center in the body. For example, the key of G is said to be grounding which works with the root Charka. Modulating then into the key of E for the sacral Charka, the composition would move next to the key of F, and so on. Other keys may be used if desired.
3) Beats/Syncopation—Sensory Component 3. Binaural beats are generated that bring the user's brain waves from its active Beta state (13-60 pulses per second) to a mental and physical relaxed Alpha state (7-13 pulses per second). There will be a frequency differentiation to create this experience. If the system transmits 22 hertz in the left ear and 30 hertz in the right ear, the brain interprets this to be 8 hertz.
4) Sound Waves—Sensory Component 4. Sound Wave waves are provided or generated which may be audible or inaudible to the human ear and provide therapeutic, relaxation or healing effects in the body. Any sound wave frequencies that provide the desired effects on the body may be used if desired.
5) Images/Video—Sensory Component 5. A visual experience using an image, painting or mural, such as the graphic 1900 shown in FIG. 19, may appear on the GUI of the device 24, e.g., having seven (7) colors and seven (7) ancient Sanskrit symbols and then animating the colors and symbols in the image to enhance the visual experience in synchronization with the energy center being described in the script. For example, when the script is on the “crown” energy center (or chakra or Reiki step) the violet image of the Sanskrit symbol (or other violet image) may get brighter, or larger or pulsate in size and/or brightness, attracting and focusing the user on that energy center for greater depth of focus and concentration.

Other scripts, music/sounds, beats, sound waves, and images/video may be used if desired, provided it provides the functions described herein.

Referring to FIGS. 18A,18B,18C, and 18D, collectively is an example scripts/words text and corresponding example GUI images file (with description) 1800,1810,1820,1830, respectively, for each of the Reiki steps (or chakras or energy centers). It also includes an introduction or “intro” portion and an “outro” or exit portion with corresponding images that may be used, if desired. In particular, FIGS. 18A-18D show an Introduction (FIG. 18A), Reiki steps 1-3 (FIG. 18B), Reiki steps 4-6 (FIG. 18C), and Reiki step 7 and Outro/Ending (FIG. 18D). The text associated with each step is an example of scripts that may be spoken as part of the sensory component 1 (script/words) for each Reiki step. The associated image(s), is an example of images that may be displayed on the display of the device 24 to the patient/user for each Reiki step (and an Introduction and an Outro/Ending).

In some embodiments, the visual experience may start with a violet Sanskrit (such as that shown in FIGS. 18A-18D), or other violet-colored image and then zoom into an animation of the human body and which shows how the energy center connects to or affects the body. In some embodiments, the visualization may show an example of the disease state in the body being attacked by the energy center for healing purposes. In that case, the visualization may show what is happening (or what is desired to be happening) in the body at a cellular and/or vascular level. For example, the visualization may show the user travelling through, along and/or into veins, blood vessels, blood cells, nerves, skin, muscles, tendons, ligaments, organs, valves, bones, joints, cartilage, bone marrow, fluids, neurons, synapses, or any other area of the body affected by the disease or disorder desired to be treated and using various energy medicine techniques to remove or reduce or minimize it. Any other colors or visualizations may be used if desired to obtain the desires response or results from the patient/client/user.

In some embodiments, Treatment App 12 (FIG. 1) may be located on a remote server and the A/V device, e.g., a smartphone or tablet or the like, may have a corresponding Device Treatment App 102 loaded on the device/smartphone 24 that may act as a “front end” interface with the user, that receives the input data from the patient/client/user and sends the input data to the Treatment App 12 located on a remote network server, e.g., the Treatment Application Server 108 (FIG. 1A). The Treatment App 12 may then perform the calculations using the data received from the device/smartphone 24, create the digital A/V treatment experience file (as described herein) and send it to the A/V device/smartphone Device Treatment App 102 for viewing by the patient/client/user. In some embodiments, the Treatment App 12 may be located on a remote server and the user logs into a website, enters the user's information and launches the treatment session, which is sent to the desired A/V device specified by the user, or it sends the user an email with a link to launch the treatment session from the desired A/V device when the user is ready.

Instead of sending the full treatment experience file from the Treatment App 12 to the A/V device 24 to be played or displayed, the digital A/V treatment file 22 could be run on a remote server (or cloud server), e.g., the Treatment Application Server 108 or other sever, and the digital A/V content streamed in real-time on-line over the internet (or other network) to the A/V device 24. In some embodiments, the Treatment App 12 could send pointers, labels or addresses to the A/V device 24 of the treatment file (or files) to be uploaded (or streamed in parts or segments) and played as part of the treatment experience. When audio/video streaming is used, the present disclosure may be used with any form of audio/video content streaming technology, streaming TV or media players, such as Roku®, Apple TV®, Google/Android TV® (Nvidia® shield), Amazon Fire® TV stick, and the like, or may be streamed to smartphones, tablets, PCs, laptops, e-readers, or virtual reality or gaming platforms (as discussed herein), or device that provides similar functions.

The user may obtain the Device Treatment App 102 for the user's smartphone or other A/V device 24 from an on-line App store or the like. The Treatment App 12 may allow the user to customize the local App 102 settings and options, such as brightness, sound levels, to optimize the audio/visual treatment experience. The service may be paid for electronically on-line by the user at the time of purchasing the Treatment Application 12 or the user may pay electronically a monthly or annual subscription fee or a use-based access fee for each time a treatment session is provided to the user.

The Treatment App 12 may also provide data to the user's doctor(s) or health insurance company, or other service provider or vendor, regarding the use of the Treatment App (e.g., when and how often treatment is provided to the user) and the results/outcomes data regarding the results or outcomes of the treatment for doctor follow-up purposes, insurance claim collection, insurance premium calculations/discounts, or other medical/insurance purposes.

The Treatment App 12 may also prompt the patient/client/user for results/outcomes data over a predetermined period of time after a given treatment session has ended, to continue to collect results/outcomes data from the patient/client/user. This may be done by e-mail, text, automated call, or other digital communications or alerts platforms. Also, the Treatment App may have scheduling features that automatically creates a schedule of treatment sessions (or appointments) for the user (or allows the user to create his/her own schedule within certain required parameters), and corresponding digital email, text, or automated call reminders or alerts. The Treatment App 12 may be launched automatically, e.g., when a scheduled treatment session is scheduled to occur, or on demand by the user. It may also provide a grace (or snooze) period within which the treatments should be held to maintain the proper treatment results/outcome schedule, e.g., it may provide an alert which tells the user a predetermined time (e.g., 15 min.) in advance of a treatment session start time, and that the user should be ready to start a session in that time frame (e.g., 15 min.).

Also, although the disclosure has been described as being used for Reiki, the present disclosure maybe used with any form of energy healing, guided meditation, hypnosis treatment, or other types of CAM (Complementary and Alternative Medicine) treatments capable of being delivered via an audio/visual experience.

The Treatment Experience App (or Treatment App or Virtual Energy Medicine app) 12, including the corresponding Device Treatment App 102 in the A/V Device/smartphone 24 that interacts with the Treatment Experience App 12, of the present disclosure, provides an energy medicine experience that can be self-administered and digitally delivered anytime, anywhere, by people who are in pain or otherwise need treatment for a disease or disorder. It may be delivered through any electronic medium that provides the functions described herein. It empowers the patient/client/user to play a proactive role in his/her own recovery and complements western or traditional medicine approaches/treatment. In addition, it learns and adapts the treatment to the patient based on results/outcomes from the current patient and other patients around the world, and can be updated in real-time. It allows the user to select their physical and emotional ailments and the application automatically modifies the treatment file or program to give more attention to area(s) of need, and less attention to others, as appropriate. It also captures and saves data from the users to build a “big data” database of results/outcomes to enhance and optimize treatment adjustment decisions.

The system described herein may be a computer-controlled device having the necessary electronics, computer processing power, interfaces, memory, hardware, software, firmware, logic/state machines, databases, microprocessors, communication links, displays or other visual or audio user interfaces, printing devices, and any other input/output interfaces, to provide the functions or achieve the results described herein. Except as otherwise explicitly or implicitly indicated herein, process or method steps described herein are implemented within software modules (or computer programs) executed on one or more general purpose computers. Specially designed hardware may alternatively be used to perform certain operations. In addition, computers or computer-based devices described herein may include any number of computing devices capable of performing the functions described herein, including but not limited to: tablets, laptop computers, desktop computers and the like.

Although the disclosure has been described herein using exemplary techniques, algorithms, or processes for implementing the present disclosure, it should be understood by those skilled in the art that other techniques, algorithms and processes or other combinations and sequences of the techniques, algorithms and processes described herein may be used or performed that achieve the same function(s) and result(s) described herein and which are included within the scope of the present disclosure.

Any process descriptions, steps, or blocks in process flow diagrams provided herein indicate one potential implementation, and alternate implementations are included within the scope of the preferred embodiments of the systems and methods described herein in which functions or steps may be deleted or performed out of order from that shown or discussed, including substantially concurrently or in reverse order, depending on the functionality involved, as would be understood by those reasonably skilled in the art.

It should be understood that, unless otherwise explicitly or implicitly indicated herein, any of the features, characteristics, alternatives or modifications described regarding a particular embodiment herein may also be applied, used, or incorporated with any other embodiment described herein. Also, the drawings herein are not drawn to scale, unless indicated otherwise.

Conditional language, such as, among others, “can,” “could,” “might,” or “may,” unless specifically stated otherwise, or otherwise understood within the context as used, is generally intended to convey that certain embodiments could include, but do not require, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Although the invention has been described and illustrated with respect to exemplary embodiments thereof, the foregoing and various other additions and omissions may be made therein and thereto without departing from the spirit and scope of the present disclosure.

What is claimed is:

1. A computer-implemented method, under control of one or more computing devices configured with specific computer-executable instructions, comprising:
receiving a plurality of audio sensory component digital files having at least one digital audio file type, the digital audio file type comprising: Script/Words files, Music/Tones files, Beats/Syncopations files, and Sound Waves files;
each of the audio sensory component digital files having audio sub-component digital data indicative of one or more audio sub-components corresponding to each of the audio sensory component digital files, the audio sub-components comprising:
at least one of: Script & Length, Languages, Voice Type, and Narration Style for the Script/Words files;
at least one of: Music Score & Length, Musical Keys, Instrument/Tone/Sound Type, and Rhythms/Cadence/Speeds for the Music/Tones files;
Beats Segment & Length for the Beats/Syncopations files; and
Frequency Range & Length for the Sound Waves files;
receiving user data indicative of a user's medical condition, current medical treatment, and personal characteristics, the medical condition comprising pain and the current medical treatment comprising pain medication;
providing selection factors/attributes for each of the audio sensory component digital files and corresponding audio sub-components based on the user data;
automatically selecting, based on the selection factors/attributes, an audio sensory component digital file and corresponding audio sub-component digital data from at least one of the audio file types, as selected audio sensory component digital files;
automatically combining the selected audio sensory components digital files from each selected audio file type to create a digital audio/visual meditation file;
receiving results/outcomes data indicative of results/outcomes for the user and for other users having a similar medical condition that have used the digital audio/visual meditation file;
continuously adjusting in real-time the selection factors/attributes using machine learning and the results/outcomes data; and
providing the digital audio/visual meditation file to the user.

2. The computer-implemented method for claim 1, further comprising:
receiving a plurality of images/video sensory component digital files, having at least one digital images/video file type, the digital images/video file types comprising: image files and video files;
each of the images/video sensory component digital files comprising image/video sub-component digital data indicative of one or more images/video sub-components corresponding to each of the images/video sensory component digital files, the images/video sub-components comprising:
at least one of Images, Brightness, and Special Effects Images for the image files; and
at least one of Video & Length, Brightness, and Special Effects Video for the video files;
providing the selection factors/attributes for each of the images/video sensory components and sub-components based on the user data;
automatically selecting, based on the selection factors/attributes, one or more of the images/video sensory components digital files with corresponding images/video sub-component digital data, as selected images/video sensory component digital files; and
automatically combining the selected images/video sensory component digital files with the selected audio sensory component digital files, to create the digital audio/video meditation file.

3. The computer-implemented method of claim 1, wherein the results/outcomes data comprises data indicative of at least one of: short term results/outcomes, long term results/outcomes, and whether the results/outcome data has been verified; wherein the short term results/outcomes comprises current treatment short term results from user, and wherein the long term results/outcomes comprises, at least one of: user assessment, doctor assessment, hospital admission/discharge/re-admission data, insurance data, pain medication prescription data, and measurement data.

4. The computer-implemented method of claim 1, further comprising adjusting the results/outcomes data based on whether the results/outcomes data has been verified and when not verified, adjusting the results/outcomes data based on non-objective factors.

5. The computer-implemented method of claim 1, wherein the selection factors/attributes for each of the sensory components and sub-components is based on a factors/attributes model.

6. The computer-implemented method of claim 1, further comprising providing a factors/attributes map indicative of the selection factors/attributes for each of the components and sub-components.

7. The computer-implemented method of claim 1, wherein the digital audio/visual meditation file comprises at least one of: guided meditation, audio/visual therapy, energy medicine treatment, and reiki/energy therapy.

8. The computer-implemented method of claim 1, wherein the user data comprises data indicative of at least one of: “Hard” Facts, “Soft” Facts, Medical Condition, Current Medical Treatment, Current CAM Medical Treatment, Environment, and Requirements/Desired Outcomes, and wherein “Hard” Facts comprises at least one of: gender, age, height, weight, birth place, culture/ethnicity, DNA map/markers, educational level/IQ, and CAM treatment history, and wherein the user “Soft” Facts comprises at least one of: suggestibility, teachability, irritability, patience, personality trait, and personality type.

9. The computer-implemented method of claim 1, further comprising repeating the selecting and the combining to create a plurality of digital audio/visual meditation files, and delivering the plurality of digital audio/visual meditation files to a user device based on a predetermined digital audio/visual meditation file delivery schedule for a single stage or a multi-stage treatment plan.

10. The computer-implemented method of claim 1, wherein the audio sub-components further comprises:
at least one of: Speed and Volume/Special Effects, for the Scripts/Words files, for the Music/Tones files, and for the Beats/Syncopation files; and
at least one of: Speed/Sweep and Amplitude/Special Effects for the Sound Wave files.

11. The computer-implemented method of claim 1, wherein the current medical treatment further comprises at least one of: chemotherapy, radiation, and surgery.

12. The computer-implemented method of claim 1, wherein the personal characteristics comprises data indicative of user “Hard” Facts, comprising at least two of: gender, age, height, weight, birth place, and culture/ethnicity of the user, and further comprising at least one of: DNA map/markers, educational level/IQ, and CAM treatment history of the user.

13. The computer-implemented method of claim 1, wherein the personal characteristics comprises data indicative of user “Soft” Facts, wherein the user “Soft” Facts comprises at least one of: suggestibility, teachability, irritability, patience, personality trait, and personality type.

14. The computer-implemented method of claim 1, wherein the selecting of the audio sensory component digital files and corresponding audio sub-components are based on the pain medication.

15. The computer-implemented method of claim 1, wherein the results/outcomes data comprises pain medication prescription data and the selecting of the audio sensory component digital files and corresponding audio sub-components are based on the pain medication prescription data.

16. The computer-implemented method of claim 1, wherein the results/outcomes data comprises hospital admission/discharge/re-admission data and the selecting of the audio sensory component digital files and corresponding audio sub-components are based on the hospital admission/discharge/re-admission data.

17. A computer system having a computer comprising at least one computer processor and a memory, wherein the computer is adapted to execute a computer program stored in the memory which causes the computer system to perform a method, comprising:
receiving a plurality of audio sensory component digital files having at least one digital audio file type, the digital audio file type comprising: Script/Words files and Music/Tones files;
each of the audio sensory component digital files comprising audio sub-component digital data indicative of one or more audio sub-components corresponding to each of the audio sensory component digital files, the audio sub-component digital data comprising: Script & Length, Languages, Voice Type, Narration Style, Speed, Music Score & Length, Musical Keys, and Instrument/Tone/Sound Type;
receiving user data indicative of a user's medical condition, current medical treatment, personal characteristics, the medical condition comprising at least two of: pain, pain location, pain type and pain severity;
providing selection factors/attributes for each of the audio sensory component digital files and corresponding audio sub-components based on the user data;
automatically selecting, based on the selection factors/attributes, the audio sensory component digital files and corresponding audio sub-component digital data, as selected audio sensory component digital files;
automatically combining the selected audio sensory components digital files from each selected audio file type to create a digital audio/visual meditation file;
receiving results/outcomes data indicative of treatment results/outcomes for the user and for other users having a similar medical condition that have used the digital audio/visual meditation file;
continuously adjusting in real-time the selection factors/attributes using machine learning and the results/outcomes data; and
digitally delivering the digital audio/visual meditation file to a user device, based on a predetermined file delivery schedule, for use by the user.

18. The computer system of claim 17, wherein the providing the selection factors/attributes for each of the sensory components and sub-components is based on a factors/attributes model.

19. The computer system of claim 17, further comprising providing a factors/attributes map indicative of the factors/attributes for each of the components and sub-components.

20. The computer system of claim 17, wherein the continuously adjusting further comprises continuously adjusting a factors/attributes model.

21. The computer system method of claim 17, wherein the audio sensory component digital files types further comprises Beats/Syncopation files, and wherein the audio sub-components further comprises at least one of: Rhythms/Cadence/Speeds and Beats Segment & Length for the Beats/Syncopation files.

22. The computer system method of claim 17, wherein the audio sensory component digital files types further comprises Sound Waves files, and wherein the audio sub-components further comprises at least one of: Frequency Range & Length and Speed/Sweep for the Sounds Wave files.

23. The computer system method of claim 17, wherein the current medical treatment comprises at least one of: chemotherapy, radiation, surgery, and pain medication.

24. The computer system method of claim 17, wherein the personal characteristics comprises data indicative of user “Hard” Facts, comprising at least two of: gender, age, height, weight, birth place, and culture/ethnicity of the user, and further comprising at least one of: DNA map/markers, educational level/IQ, and CAM treatment history of the user.

25. The computer system method of claim 17, wherein the current medical treatment comprises pain medication and the selecting of the audio sensory component digital files and corresponding audio sub-components are based on the pain medication.

26. The computer system method of claim 17, wherein the results/outcomes data comprises pain medication prescription data and the selecting of the audio sensory component digital files and corresponding audio sub-components are based on the pain medication prescription data.

27. The computer system method of claim 17, wherein the results/outcomes data comprises hospital admission/discharge/re-admission data and the selecting of the audio sensory component digital files and corresponding audio sub-components are based on the hospital admission/discharge/re-admission data.

28. A computer-implemented digital audio/visual processing method, comprising:
receiving one or more audio sensory component digital files, each of the audio sensory component digital files comprising audio sub-component digital data indicative of one or more audio sub-components;
receiving user data indicative of a user's medical condition, current medical treatment, and personal characteristics, the medical condition comprising pain and the current medical treatment comprising pain medication;
providing selection factors/attributes for each of the audio sensory component digital files and corresponding audio sub-component based on the user data;
automatically selecting, based on at least the selection factors/attributes, one or more of the audio sensory component digital files and corresponding audio sub-component digital data, as selected audio sensory component digital files;
automatically combining the selected audio sensory components digital files to create a digital audio/visual meditation file;
receiving results/outcomes data indicative of results/outcomes for the user and for other users having a similar medical condition that have used the digital audio/visual meditation file, wherein the results/outcomes data comprises at least one of: pain medication prescription data and hospital admission/discharge/re-admission data;
continuously adjusting in real-time the selection factors/attributes using machine learning and the results/outcomes data; and
providing the digital audio/visual meditation file to a user device, for use by the user.

29. The computer-implemented method of claim 28, further comprising:
receiving a plurality of images/video sensory component digital files, having at least one digital images/video file type, the digital images/video file types comprising: image files and video files;
each of the images/video sensory component digital files comprising image/video sub-component digital data indicative of one or more images/video sub-components corresponding to each of the images/video sensory component digital files, the images/video sub-components comprising:
at least one of: Images, Brightness, and Special Effects Images for the image files; and
at least one of: Video & Length, Brightness, and Special Effects Video for the video files;
providing the selection factors/attributes for each of the images/video sensory components and sub-components based on the user data;
automatically selecting, based on the selection factors/attributes, one or more of the images/video sensory components digital files with corresponding images/video sub-component digital data, as selected images/video sensory component digital files; and
automatically combining the selected images/video sensory component digital files with the selected audio sensory component digital files, to create the digital audio/visual meditation file.

30. The computer-implemented method of claim 28,
wherein the plurality of audio sensory component digital files have at least one digital audio file type, the digital audio file type comprising: Script/Words files, Music/Tones files, Beats/Syncopations files, and Sound Waves files; and
wherein the audio sub-components comprises:
at least one of Script & Length, Languages, Voice Type, and Narration Style for the Script/Words files;
at least one of Music Score & Length, Musical Keys, Instrument/Tone/Sound Type, and Rhythms/Cadence/Speeds for the Music/Tones files;
Beats Segment & Length for the Beats/Syncopations files; and
Frequency Range & Length for the Sound Waves files.

31. The computer-implemented method of claim 28, wherein the user's medical condition further comprises at least one of: a disease, an illness, a morbidity, a disorder, a habit, and an addiction.

32. The computer-implemented method of claim 28, wherein the current medical treatment further comprises at least one of: chemotherapy, radiation, and surgery.

33. The computer-implemented method of claim 28, wherein the personal characteristics comprises data indicative of user “Hard” Facts, comprising at least two of: gender, age, height, weight, birth place, and culture/ethnicity of the user, and further comprising at least one of: DNA map/markers, educational level/IQ, and CAM treatment history of the user.

34. The computer-implemented method of claim 28, wherein the selecting of the audio sensory component digital files and corresponding audio sub-components are based on the pain medication.

35. The computer-implemented method of claim 28, wherein the results/outcomes data comprises pain medication prescription data and the selecting of the audio sensory component digital files and corresponding audio sub-components are based on the pain medication prescription data.

36. The computer-implemented method of claim 28, wherein the results/outcomes data comprises hospital admission/discharge/re-admission data and the selecting of the audio sensory component digital files and corresponding audio sub-components are based on the hospital admission/discharge/re-admission data.

37. The computer-implemented method of claim 28, wherein the pain medication comprises opiates.
